(19) United States
(12) Patent Application Publication
(10) Pub. No.: US 2018/0042868 A1
(43) Pub. Date: Feb. 15, 2018

(54) TAMPER RESISTANT FORMULATION OF EPHEDRINE AND ITS DERIVATIVES

(71) Applicant: GRÜNENTHAL GMBH, Aachen (DE)

(72) Inventors: Carmen STOMBERG, Korschenbroich (DE); Klaus WENING, Koln (DE); Sebastian SCHWIER, Aachen (DE)

(73) Assignee: GRÜNENTHAL GMBH, Aachen (DE)

(21) Appl. No.: 15/674,613

(22) Filed: Aug. 11, 2017

(30) Foreign Application Priority Data
Aug. 12, 2016 (EP) 16183922.0
Nov. 25, 2016 (EP) 16200767.8
May 30, 2017 (EP) 17173383.5

(51) Int. Cl. A61K 31/137; A61K 9/00; A61K 47/38; A61K 47/32; A61K 47/10; A61K 47/22

(52) U.S. Cl. CPC A61K 31/137; A61K 47/10; A61K 47/22; A61K 47/38; A61K 47/32; A61K 9/0053

(57) ABSTRACT

A pharmaceutical dosage form having a breaking strength of at least 300 N and comprising an ephedrine component selected from the group consisting of ephedrine, pseudoephedrine and the physiologically acceptable salts thereof, wherein the weight content of the ephedrine component is within the range of from 0.1 to 60 wt.-%, relative to the total weight of the pharmaceutical dosage form.

This application claims priority of European Patent Applications Nos. EP 16183922.0 filed on Aug. 12, 2016, EP 16200767.8 filed on Nov. 25, 2016, and EP 17173383.5 filed on May 30, 2017, the entire contents of which patent applications are hereby incorporated herein by reference.

The invention relates to a pharmaceutical dosage form having a breaking strength of at least 300 N and comprising an ephedrine component selected from the group consisting of ephedrine, pseudoephedrine and the physiologically acceptable salts thereof, wherein the weight content of the ephedrine component is within the range of from 0.1 to 60 wt.-%, relative to the total weight of the pharmaceutical dosage form. The pharmaceutical dosage form according to the invention provides resistance against tampering, particularly against conversion of the ephedrine component into methamphetamine (also known as e.g. “Crystal Meth”, “Meth”, “Crystal”, “Yaba”, “Crank” or “Ice”).

Pseudoephedrine is a stimulant and decongestant. It reduces tissue hyperemia, edema, and nasal congestion commonly associated with colds or allergies. Other beneficial effects may include increasing the drainage of sinus secretions, and opening of obstructed Eustachian tubes.

Pseudoephedrine and ephedrine are diastereomers. Pseudoephedrine has threo-configuration and ephedrine has erythro-configuration. Both diastereomers exist in form of two enantiomers each:

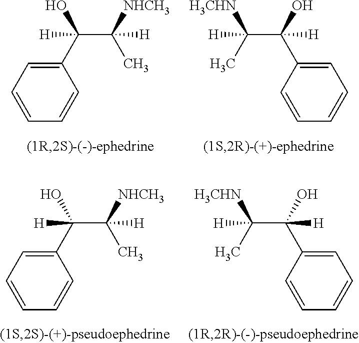

Due to its widespread abuse in the illicit manufacture of methamphetamine, the distribution of pseudoephedrine has been severely restricted and controlled, thereby placing undue burden on the individuals suffering from nasal congestion and other related discomforts from being able to readily obtain this highly effective medication.

Unfortunately, such illicit manufacture of methamphetamine from ephedrine or pseudoephedrine also results in serious bodily injury or death to the victims. Therefore, development of an effective abuse-deterrent composition comprising pseudoephedrine and substantially suppresses or blocks the chemical conversion of pseudoephedrine or ephedrine to methamphetamine remains an urgent unmet need.

Methamphetamine ((RS)—N-methyl-1-phenylpropan-2-amine) also exists in form of two enantiomers but has only a single stereocenter.

Illicit manufacture of methamphetamine from pseudoephedrine and ephedrine has been accomplished by numerous methods that involve reduction of pseudoephedrine or ephedrine with various reducing agents including lithium, zinc, and phosphorous.

Two different approaches may be roughly distinguished from one another. In one pot approaches, the dosage form, optionally after pulverization, is subjected to chemical conversion in a suitable solvent with suitable chemicals. In two pot approaches, the pseudoephedrine/ephedrine is first extracted from the dosage form, optionally after pulverization, and the thus obtained extract is subsequently subjected to chemical conversion in a suitable solvent with suitable chemicals.

A popular one pot method is the so-called “soda-bottle shake and bake” procedure (for the purpose of the specification also referred to as “shake and bake one pot procedure”) using lithium and ammonium nitrate. The reagents and the common solvents such as ether, toluene, light petroleum, ammonia, hydrochloric acid, hydriodic acid, sodium hydroxide, and the like are readily accessible to the illicit manufacturers (see also R. Turkington, Chemicals Used For Illegal Purposes, A Guide for First responders to Identify Explosives, Recreational Drugs, and Poisons, John Wiley & Sons 2010, page 247).

Various solid pharmaceutical dosage formulations have been introduced which are said to physically impede the extraction of pseudoephedrine from such formulations (e.g., Tarex®, Sudafed® and Nexafed®). ZephrexD® is a meth-resistant form of pseudoephedrine that becomes gooey when heated.

US 2004 0049079 relates to a method of inhibiting or preventing the use of anhydrous ammonia as a solvent in a dissolving metal reduction process comprises adding to anhydrous ammonia a chemical reagent which is capable of scavenging solvated electrons generated when alkali or alkaline earth metal is dissolved in the anhydrous ammonia, the chemical reagent being added to the anhydrous ammonia such that when alkali metal is dissolved in the anhydrous ammonia containing the chemical reagent and thereafter ephedrine, pseudoephedrine or combination thereof is introduced to the anhydrous ammonia to produce a reaction product, the methamphetamine yield in the reaction product is below 50%, preferably below 10%, and more preferably below 1%.

US 2008 0260836 discloses films that comprise a first polymer and a second polymer having a solubility temperature lower than that of the first polymer; wherein the breaking strength of the film is greater than about 750 psi (5,171 kPa).

US 2008 0311187 relates to a pharmaceutical dosage form comprising a physiologically effective amount of a physiologically active substance (A), a synthetic, semi-synthetic or natural polymer (C), optionally one or more physiologically acceptable auxiliary substances (B) and optionally a synthetic, semi-synthetic or natural wax (D), wherein the pharmaceutical dosage form exhibits a resistance to crushing of at least 400 N and wherein under physiological conditions the release of the physiologically active substances (A) from the pharmaceutical dosage form is at least partially delayed.

US 2009 0004267 discloses a multiparticulate pharmaceutical dosage form formulated to make misuse more difficult containing least one active substance with potential for misuse (A), at least one synthetic or natural polymer (C), optionally at least one natural, semi-synthetic or synthetic wax (D), at least one disintegrant (E) and optionally one or more additional physiologically compatible excipients (B), wherein the individual particles of the pharmaceutical dosage form display a breaking strength of at least 500 N and a release of active substance of at least 75% after 45 minutes measured according to Ph. Eur. in the paddle mixer with sinker in 600 ml of aqueous buffer solution with a pH value of 1.2 at 37° C. and 75 rpm.

US 2009 0202634 and WO 2009/092601 relate to a pharmaceutical dosage form, preferably with controlled release of a pharmacologically active compound (A) contained therein, the pharmaceutical dosage form very preferably being tamper-resistant and most preferably having a breaking strength B1 of at least 500 N in direction of extension E1 and having a breaking strength B2 of less than 500 N in direction of extension E2.

US 2013 225625 relates to a pharmaceutical dosage form having a breaking strength of at least 500 N and comprising a pharmacologically active compound, a polyalkylene oxide having an average molecular weight of at least 200,000 g/mol, and a nonionic surfactant; wherein the content of the polyalkylene oxide is within the range of from 20 to 75 wt.-%, based on the total weight of the pharmaceutical dosage form.

US 2014 0010874 discloses in certain embodiments a solid oral pharmaceutical dosage form comprising: (a) an inert tamper resistant core; and (b) a coating surrounding the core, the coating comprising an active agent.

US 2014 356426 relates to a tamper-resistant pharmaceutical dosage form comprising one or more particles, wherein each of said one or more particles comprises a pharmacologically active ingredient and a physiologically acceptable polymer; has a breaking strength of at least 300 N; has a weight of at least 2 mg; and optionally, comprises a film-coating; wherein the total weight of the pharmaceutical dosage form is greater than the total weight of said one or more particles.

US 2015 064250 provides a tamper-resistant dosage form including a therapeutic agent-substrate complex embedded in a thermo-formable matrix; such that the complex includes at least one therapeutic agent bound to at least one substrate to form the therapeutic agent-substrate complex. The at least one substrate is being selected from the group consisting of a polyelectrolyte, an organic counter-ion, a pharmacologically inert organic component of a prodrug, an inclusion compound and an inorganic adsorbent; and the thermo-formable matrix includes one or more thermoplastic polymers and optionally at least one pharmaceutical additive.

US 2016 0089439 relates to ephedrine or pseudoephedrine compositions containing biocompatible organoleptic (food flavoring) excipients that would prevent the illicit manufacture of methamphetamine from ephedrine or pseudoephedrine.

U.S. Pat. No. 8,901,113 relates to methods and compositions to deter abuse of pharmaceutical products (e.g., orally administered pharmaceutical products) including but not limited to immediate release, sustained or extended release and delayed release formulations for drugs subject to abuse comprising at least 10% by weight hydroxypropylcellulose; polyethylene oxide; and a disintegrant selected from the group consisting of crospovidone, sodium starch glycolate and croscarmellose sodium; wherein the ratio of hydroxypropylcellulose to polyethylene oxide on a weight basis is between about 10:1 and 1:10.

The concepts of the prior art for preventing illicit manufacture of methamphetamine from ephedrine or pseudoephedrine are not satisfactory in every respect and there is a demand for improvement.

It is an object of the invention to provide pharmaceutical dosage forms that contain ephedrine, pseudoephedrine or a physiologically acceptable salt thereof and that have advantages compared to the prior art, particularly with respect to the prevention of the illicit manufacture of methamphetamine.

This object has been achieved by the subject-matter of the patent claims.

It has been surprisingly found that pharmaceutical dosage forms can be provided that contain ephedrine, pseudoephedrine or the physiologically acceptable salts thereof and that substantially impede if not fully prevent chemical conversion of the ephedrine or pseudoephedrine into methamphetamine, especially when following the particularly popular “shake and bake one pot” procedure. Further, it has been surprisingly found that pharmaceutical dosage forms can be provided that contain ephedrine, pseudoephedrine or the physiologically acceptable salts thereof and that substantially impede chemical conversion of the ephedrine or pseudoephedrine into methamphetamine after extraction of ephedrine or pseudoephedrine.

Still further, it has been surprisingly found that the above impediments remain even if mechanical disruption of the pharmaceutical dosage form can be achieved at least to a certain degree, in spite of its increased mechanical strength, e.g. even if a person intending illicit misuse has access to suitable equipment.

Yet further, it has been surprisingly found that antioxidants, especially α-tocopherol, may suppress chemical conversion of the ephedrine or pseudoephedrine into methamphetamine.

Furthermore, it has been surprisingly found that polyvinylpyrrolidone (PVP) and croscarmellose, particularly when being combined with one another, provide pharmaceutical dosage forms that yield coarse particles upon grinding, if the pharmaceutical dosage forms can be ground at all. Yet further, when trying to extract the ephedrine or pseudoephedrine from such coarse particles with water and diethylether, a stabile emulsion is formed, i.e. the ether phase does not separate from the water phase so that extraction by means of a separatory funnel is not at all or at least hardly possible.

Moreover, there is indication that polyvinylpyrrolidone (PVP) exhibits a certain solubility in those solvents that are conventionally used by abusers in attempts of extracting ephedrine or pseudoephedrine from pharmaceutical dosage forms or attempts of chemically converting ephedrine or pseudoephedrine to methamphetamine. This results in a desirable impurity of the obtained intermediate extract or yielded product thus contaminating the intermediate extract or final product and impeding further abuse and administration thereof, respectively.

A first aspect of the invention relates to a pharmaceutical dosage form having a breaking strength of at least 300 N and comprising an ephedrine component selected from the group consisting of ephedrine, pseudoephedrine and the physiologically acceptable salts thereof, preferably pseudoephedrine hydrochloride or pseudoephedrine sulfate, wherein the weight content of the ephedrine component is within the range of from 0.1 to 60 wt.-%, relative to the total weight of the pharmaceutical dosage form.

For the purpose of the description, unless expressly stated otherwise, all percentages are weight percent (wt.-%).

For the purpose of the description, unless expressly stated otherwise, all values with regard to the content of the ephedrine component (e.g. in mg or in wt.-%) and of any combined pharmacologically active ingredients, if any, are expressed as weight equivalents with regard to the free base of the ephedrine component (i.e. ephedrine free base and pseudoephedrine free base, respectively) and of the combined pharmacologically active ingredients, respectively.

The pharmaceutical dosage form according to the invention comprises an ephedrine component selected from the group consisting of ephedrine, pseudoephedrine and the physiologically acceptable salts thereof.

In a preferred embodiment, the pharmaceutical dosage form contains ephedrine component as the sole pharmacologically active ingredient.

In another preferred embodiment, the pharmaceutical dosage form contains a combination of ephedrine component with one or more pharmacologically active ingredients.

The ephedrine component may be present in form of a physiologically acceptable salt, e.g. physiologically acceptable acid addition salt. Preferred salts include but are not limited to hydrochlorides and sulfates. Preferably, the ephedrine component comprises or essentially consists of pseudoephedrine hydrochloride or pseudoephedrine sulfate.

Physiologically acceptable salts comprise the acid addition salt forms which can conveniently be obtained by treating the base form of the ephedrine component with appropriate organic and inorganic acids. The salt also comprises the hydrates and solvent addition forms which the ephedrine component is able to form. Examples of such forms are e.g. hydrates, alcoholates and the like.

The ephedrine component is present in the pharmaceutical dosage form in a therapeutically effective amount. The amount that constitutes a therapeutically effective amount varies according to the active ingredients being used, the condition being treated, the severity of said condition, the patient being treated, and whether the pharmaceutical dosage form is designed for an immediate or retarded release.

The weight content of the ephedrine component is within the range of from 0.1 to 60 wt.-%, relative to the total weight of the pharmaceutical dosage form. Preferably, the weight content of the ephedrine component is within the range of from 15 to 45 wt.-%, relative to the total weight of the pharmaceutical dosage form.

Preferably, the weight content of the ephedrine component is within the range of 30±18 wt.-%, more preferably 30±15 wt.-%, still more preferably 30±12 wt.-%, yet more preferably 30±9 wt.-%, even more preferably 30±6 wt.-%, and most preferably 30±3 wt.-%, based on the total weight of the pharmaceutical dosage form.

The absolute dose of the ephedrine component in the pharmaceutical dosage form is not limited. The dose of the ephedrine component which is adapted for administration preferably is in the range of 0.1 mg to 500 mg, more preferably in the range of 1.0 mg to 400 mg, even more preferably in the range of 5.0 mg to 300 mg, and most preferably in the range of 10 mg to 250 mg.

In a preferred embodiment, the ephedrine component is contained in the pharmaceutical dosage form in an amount of 7.5±5 mg, 10±5 mg, 20±5 mg, 30±5 mg, 40±5 mg, 50±5 mg, 60±5 mg, 70±5 mg, 80±5 mg, 90±5 mg, 100±5 mg, 110±5 mg, 120±5 mg, 130±5, 140±5 mg, 150±5 mg, 160±5 mg, 170±5 mg, 180±5 mg, 190±5 mg, 200±5 mg, 210±5 mg, 220±5 mg, 230±5 mg, 240±5 mg, 250±5 mg, 260±5 mg, 270±5 mg, 280±5 mg, 290±5 mg, or 300±5 mg.

Preferred combinations of the ephedrine component with one or more other pharmacologically active ingredients include but are not limited to combinations of pseudoephedrine or physiologically acceptable salts thereof with acrivastine, azatadine maleate, brompheniramine maleate, chlorpheniramine, chlorpheniramine maleate, cetirizine hydrochloride, clemastine fumarate, codeine phosphate, desloratadine, dexbromopheniramine maleate, dextromethorphan hydrobromide, diphenhydramine hydrochloride, fexofenadine hydrochloride, guaifenesin, hydrocodone bitartrate, ibuprofen, loratadine, naproxen sodium, paracetamol, and/or triprolidine hydrochloride. Such binary or ternary combinations are commercially available as formulations in conventional pharmaceutical dosage forms.

The ephedrine component and the one or more other pharmacologically active ingredients may be released from the pharmaceutical dosage form according to the invention by the same or different release kinetics.

In a preferred embodiment, the ephedrine component as well as the one or more other pharmacologically active ingredients are released from the pharmaceutical dosage form according to an immediate release profile.

In another preferred embodiment, the ephedrine component as well as the one or more other pharmacologically active ingredients are released from the pharmaceutical dosage form according to a prolonged release profile.

In still another preferred embodiment, the ephedrine component is released from the pharmaceutical dosage form according to a prolonged release profile, whereas the one or more other pharmacologically active ingredients are released from the pharmaceutical dosage form according to an immediate release profile.

In yet another preferred embodiment, the ephedrine component is released from the pharmaceutical dosage form according to an immediate release profile, whereas the one or more other pharmacologically active ingredients are released from the pharmaceutical dosage form according to a prolonged release profile.

Preferred combinations of pseudoephedrine or physiologically acceptable salts thereof with triprolidine or physiologically acceptable salts thereof, preferably tripolidine hydrochloride, according to the invention include but are not limited to combinations of pseudoephedrine, preferably in form of the hydrochloride salt or sulfate salt thereof, preferably at dosages of e.g. about 30 mg, about 60 mg, about 120 mg, or about 240 mg, with triprolidine or physiologically acceptable salts thereof, preferably, tripolidine hydrochloride, preferably at dosages of e.g. about 2.5 mg.

Preferred combinations of pseudoephedrine or physiologically acceptable salts thereof with naproxen or physiologically acceptable salts thereof, preferably naproxen sodium, according to the invention include but are not limited to combinations of pseudoephedrine, preferably in form of the hydrochloride salt or sulfate salt thereof, preferably at dosages of e.g. about 30 mg, about 60 mg, about 120 mg, or about 240 mg, with naproxen or physiologically acceptable salts thereof, preferably naproxen sodium, preferably at dosages of e.g. about 240 mg.

Preferred combinations of pseudoephedrine or physiologically acceptable salts thereof with fexofenadine or physiologically acceptable salts thereof, preferably fexofenadine hydrochloride, according to the invention include but are not limited to combinations of pseudoephedrine, preferably in form of the hydrochloride salt or sulfate salt thereof, preferably at dosages of e.g. about 30 mg, about 60 mg, about 120 mg, or about 240 mg, with fexofenadine or physiologically acceptable salts thereof, preferably fexofenadine hydrochloride, preferably at dosages of e.g. about 60 mg.

Preferred combinations of pseudoephedrine or physiologically acceptable salts thereof with loratadine or physiologically acceptable salts thereof according to the invention include but are not limited to combinations of pseudoephedrine, preferably in form of the hydrochloride salt or sulfate salt thereof, preferably at dosages of e.g. about 30 mg, about 60 mg, about 120 mg, or about 240 mg, with loratadine or physiologically acceptable salts thereof, preferably at dosages of e.g. about 5 mg or about 10 mg.

Preferred combinations of pseudoephedrine or physiologically acceptable salts thereof with acrivastine or physiologically acceptable salts thereof according to the invention include but are not limited to combinations of pseudoephedrine, preferably in form of the hydrochloride salt or sulfate salt thereof, preferably at dosages of e.g. about 30 mg, about 60 mg, about 120 mg, or about 240 mg, with acrivastine or physiologically acceptable salts thereof, preferably at dosages of e.g. about 8 mg.

Preferred combinations of pseudoephedrine or physiologically acceptable salts thereof with cetirizine or physiologically acceptable salts thereof, preferably cetirizine hydrochloride, according to the invention include but are not limited to combinations of pseudoephedrine, preferably in form of the hydrochloride salt or sulfate salt thereof, preferably at dosages of e.g. about 30 mg, about 60 mg, about 120 mg, or about 240 mg, with cetirizine or physiologically acceptable salts thereof, preferably cetirizine hydrochloride, preferably at dosages of e.g. about 5 mg.

Preferred combinations of pseudoephedrine or physiologically acceptable salts thereof with chlorpheniramine or physiologically acceptable salts thereof, preferably chlorpheniramine maleate, according to the invention include but are not limited to combinations of pseudoephedrine, preferably in form of the hydrochloride salt or sulfate salt thereof, preferably at dosages of e.g. about 30 mg, about 60 mg, about 120 mg, or about 240 mg, with chlorpheniramine or physiologically acceptable salts thereof, preferably chlorpheniramine maleate.

Preferred combinations of pseudoephedrine or physiologically acceptable salts thereof with guaifenesin or physiologically acceptable salts thereof according to the invention include but are not limited to combinations of pseudoephedrine, preferably in form of the hydrochloride salt or sulfate salt thereof, preferably at dosages of e.g. about 30 mg, about 60 mg, about 120 mg, or about 240 mg, with guaifenesin or physiologically acceptable salts thereof, preferably at dosages of e.g. about 1200 mg.

Preferred combinations of pseudoephedrine or physiologically acceptable salts thereof with ibuprofen or physiologically acceptable salts thereof according to the invention include but are not limited to combinations of pseudoephedrine, preferably in form of the hydrochloride salt or sulfate salt thereof, preferably at dosages of e.g. about 30 mg, about 60 mg, about 120 mg, or about 240 mg, with ibuprofen or physiologically acceptable salts thereof, preferably at dosages of e.g. about 100 mg, about 200 mg, or about 400 mg.

Preferred combinations of pseudoephedrine or physiologically acceptable salts thereof with paracetamol (acetaminophen) or physiologically acceptable salts thereof according to the invention include but are not limited to combinations of pseudoephedrine, preferably in form of the hydrochloride salt or sulfate salt thereof, preferably at dosages of e.g. about 30 mg, about 60 mg, about 120 mg, or about 240 mg, with paracetamol or physiologically acceptable salts thereof, preferably at dosages of e.g. about 250 mg, about 300 mg, about 450 mg, about 500 mg, about 550 mg, about 600 mg, or about 650 mg.

Preferred combinations of pseudoephedrine or physiologically acceptable salts thereof with azatadine or physiologically acceptable salts thereof, preferably azatadine maleate, according to the invention include but are not limited to combinations of pseudoephedrine, preferably in form of the hydrochloride salt or sulfate salt thereof, preferably at dosages of e.g. about 30 mg, about 60 mg, about 120 mg, or about 240 mg, with azatadine or physiologically acceptable salts thereof, preferably azatadine maleate.

Preferred combinations of pseudoephedrine or physiologically acceptable salts thereof with brompheniramine or physiologically acceptable salts thereof, preferably brompheniramine maleate, according to the invention include but are not limited to combinations of pseudoephedrine, preferably in form of the hydrochloride salt or sulfate salt thereof, preferably at dosages of e.g. about 30 mg, about 60 mg, about 120 mg, or about 240 mg, with brompheniramine or physiologically acceptable salts thereof, preferably brompheniramine maleate.

Preferred combinations of pseudoephedrine or physiologically acceptable salts thereof with desloratadine or physiologically acceptable salts thereof according to the invention include but are not limited to combinations of pseudoephedrine, preferably in form of the hydrochloride salt or sulfate salt thereof, preferably at dosages of e.g. about 30 mg, about 60 mg, about 120 mg, or about 240 mg, with desloratadine or physiologically acceptable salts thereof.

Preferred combinations of pseudoephedrine or physiologically acceptable salts thereof with dexbrompheniramine or physiologically acceptable salts thereof, preferably dexbrompheniramine maleate, according to the invention include but are not limited to combinations of pseudoephedrine, preferably in form of the hydrochloride salt or sulfate salt thereof, preferably at dosages of e.g. about 30 mg, about 60 mg, about 120 mg, or about 240 mg, with dexbrompheniramine or physiologically acceptable salts thereof, preferably dexbrompheniramine maleate.

Preferred combinations of pseudoephedrine or physiologically acceptable salts thereof with diphenhydramine or physiologically acceptable salts thereof, preferably diphenhydramine hydrochloride, according to the invention include but are not limited to combinations of pseudoephedrine, preferably in form of the hydrochloride salt or sulfate salt thereof, preferably at dosages of e.g. about 30 mg, about 60 mg, about 120 mg, or about 240 mg, with diphenhydramine or physiologically acceptable salts thereof, preferably diphenhydramine hydrochloride.

Preferred combinations of pseudoephedrine or physiologically acceptable salts thereof with hydrocodone or physiologically acceptable salts thereof, preferably hydrocodone bitartrate, according to the invention include but are not limited to combinations of pseudoephedrine, preferably in form of the hydrochloride salt or sulfate salt thereof, preferably at dosages of e.g. about 30 mg, about 60 mg, about 120 mg, or about 240 mg, with hydrocodone or physiologically acceptable salts thereof, preferably hydrocodone bitartrate.

Preferred combinations of pseudoephedrine or physiologically acceptable salts thereof with codeine or physiologically acceptable salts thereof, preferably codeine phosphate, according to the invention include but are not limited to combinations of pseudoephedrine, preferably in form of the hydrochloride salt or sulfate salt thereof, preferably at dosages of e.g. about 30 mg, about 60 mg, about 120 mg, or about 240 mg, with codeine or physiologically acceptable salts thereof, preferably codeine phosphate.

Preferred combinations of pseudoephedrine or physiologically acceptable salts thereof with clemastine or physiologically acceptable salts thereof, preferably clemastine fumarate, according to the invention include but are not limited to combinations of pseudoephedrine, preferably in form of the hydrochloride salt or sulfate salt thereof, preferably at dosages of e.g. about 30 mg, about 60 mg, about 120 mg, or about 240 mg, with clemastine or physiologically acceptable salts thereof, preferably clemastine fumarate.

The ephedrine component is preferably present in a controlled-release matrix comprising a polyalkylene oxide, and optionally additionally comprising a cellulose ether, preferably hydroxypropylmethyl cellulose, a cross-linked polymer, preferably croscarmellose or croscarmellose sodium, and/or a binder, preferably polyvinylpyrrolidone.

Preferably, the pharmaceutical dosage form according to the invention comprises a polyalkylene oxide having a weight average molecular weight of at least 200,000 g/mol.

In a preferred embodiment, the polyalkylene oxide has a weight average molecular weight (M_{W}) or viscosity average molecular weight (M_{η}) of at least 500,000 g/mol, preferably at least 1,000,000 g/mol or at least 2,500,000 g/mol, more preferably in the range of about 1,000,000 g/mol to about 15,000,000 g/mol, and most preferably in the range of about 5,000,000 g/mol to about 10,000,000 g/mol. Suitable methods to determine M_{W} and M_{η} are known to a person skilled in the art. M_{η} is preferably determined by rheological measurements, whereas M_{W} can be determined by gel permeation chromatography (GPC).

Preferably, the molecular weight dispersity M_{w}/Mₙ of polyalkylene oxide is within the range of 2.5±2.0, more preferably 2.5±1.5, still more preferably 2.5±1.0, yet more preferably 2.5±0.8, most preferably 2.5±0.6, and in particular 2.5±0.4.

The polyalkylene oxide preferably has a viscosity at 25° C. of 30 to 17,600 cP, more preferably 55 to 17,600 cP, still more preferably 600 to 17,600 cP and most preferably 4,500 to 17,600 cP, measured in a 5 wt.-% aqueous solution using a model RVF Brookfield viscosimeter (spindle no. 2/rotational speed 2 rpm); of 400 to 4,000 cP, more preferably 400 to 800 cP or 2,000 to 4,000 cP, measured on a 2 wt.-% aqueous solution using the stated viscosimeter (spindle no. 1 or 3/rotational speed 10 rpm); or of 1,650 to 10,000 cP, more preferably 1,650 to 5,500 cP, 5,500 to 7,500 cP or 7,500 to 10,000 cP, measured on a 1 wt.-% aqueous solution using the stated viscosimeter (spindle no. 2/rotational speed 2 rpm).

Preferably, the polyalkylene oxide is selected from polymethylene oxide, polyethylene oxide and polypropylene oxide, or copolymers thereof. Preferably, the polyalkylene oxide is a polyethylene oxide.

Preferably, the weight content of the polyalkylene oxide is at least 30 wt.-%, relative to the total weight of the pharmaceutical dosage form.

The weight content of the polyalkylene oxide is preferably within the range of from 30 to 80 wt.-%, based on the total weight of the pharmaceutical dosage form. Preferably, the weight content of the polyalkylene oxide is within the range of 50±20 wt.-%, based on the total weight of the pharmaceutical dosage form. Preferably, the weight content of the polyalkylene oxide is within the range of 50±30 wt.-%, more preferably 50±27 wt.-%, still more preferably 50±24 wt.-%, yet more preferably 50±21 wt.-%, even more preferably 50±18 wt.-%, and most preferably 50±15 wt.-%, based on the total weight of the pharmaceutical dosage form.

The polyalkylene oxide may comprise a single polyalkylene oxide having a particular average molecular weight, or a mixture (blend) of different polymers, such as two, three, four or five polymers, e.g., polymers of the same chemical nature but different average molecular weight, polymers of different chemical nature but same average molecular weight, or polymers of different chemical nature as well as different molecular weight.

For the purpose of the specification, a polyalkylene glycol has a molecular weight of up to 20,000 g/mol whereas a polyalkylene oxide has a molecular weight of more than 20,000 g/mol. In a preferred embodiment, the weight average over all molecular weights of all polyalkylene oxides that are contained in the pharmaceutical dosage form is at least 200,000 g/mol. Thus, polyalkylene glycols, if any, are preferably not taken into consideration when determining the weight average molecular weight of polyalkylene oxide.

In a preferred embodiment, polyalkylene oxide is homogeneously distributed in the pharmaceutical dosage form according to the invention. Preferably, the ephedrine component and polyalkylene oxide are intimately homogeneously distributed in the pharmaceutical dosage form so that the pharmaceutical dosage form does not contain any segments where either ephedrine component is present in the absence of polyalkylene oxide or where polyalkylene oxide is present in the absence of ephedrine component.

When the pharmaceutical dosage form is film coated, the polyalkylene oxide is preferably homogeneously distributed in the core of the pharmaceutical dosage form, i.e. the film coating preferably does not contain polyalkylene oxide. Nonetheless, the film coating as such may of course contain one or more polymers, which however, preferably differ from the polyalkylene oxide contained in the core.

Preferably, the relative weight ratio of the polyalkylene oxide to the ephedrine component is within the range of from 5:1 to 1:4, more preferably from 4.5:1 to 1:3.5, still more preferably from 4:1 to 1:3, yet more preferably from 3.5:1 to 1:2.5, even more preferably from 3:1 to 1:2, most preferably from 2.5:1 to 1:1.5, and in particular from 2:1 to 1:1.

The polyalkylene oxide may be combined with one or more different polymers selected from the group consisting of polyethylene, polypropylene, polyvinyl chloride, polycarbonate, polystyrene, polyvinylpyrrolidone, poly(alk)acrylate, poly(hydroxy fatty acids), such as for example poly(3-hydroxybutyrate-co-3-hydroxyvalerate) (Biopol®), poly(hydroxyvaleric acid); polycaprolactone, polyvinyl alcohol, polyesteramide, polyethylene succinate, polylactone, polyglycolide, polyurethane, polyamide, polylactide, polyacetal (for example polysaccharides optionally with modified side chains), polylactide/glycolide, polylactone, polyglycolide, polyorthoester, polyanhydride, block polymers of polyethylene glycol and polybutylene terephthalate (Polyactive®), polyanhydride (Polifeprosan), copolymers thereof, block-copolymers thereof, and mixtures of at least two of the stated polymers, or other polymers with the above characteristics.

Preferably, the pharmaceutical dosage form according to the invention comprises an antioxidant. Preferably, the antioxidant is selected from the group consisting of ascorbic acid, salts of ascorbic acid, butylhydroxyanisole (BHA), butylhydroxytoluene (BHT), monothioglycerol, phosphorous acid, α-tocopherol, α-tocopheryl acetate, coniferyl benzoate, nordihydroguajaretic acid, gallus acid esters, and sodium bisulfate. A particularly preferred antioxidant is α-tocopherol.

Preferably, the weight content of the antioxidant, preferably α-tocopherol, is greater than 0.2 wt.-%, more preferably at least 0.3 wt.-% or at least 0.4 wt.-%, still more preferably at least 0.5 wt.-% or at least 0.6 wt.-%, yet more preferably at least 0.7 wt.-% or at least 0.8 wt.-%, even more preferably at least 0.9 wt.-% or at least 1.0 wt.-%, most preferably at least 1.1 wt.-% or at least 1.2 wt.-%, and in particular at least 1.3 wt.-% or at least 1.4 wt.-%, in each case relative to the total weight of the pharmaceutical dosage form.

Preferably, the weight content of the antioxidant is within the range of 1.00±0.95 wt.-%, based on the total weight of the pharmaceutical dosage form. Preferably, the weight content of the antioxidant is within the range of 1.5±0.6 wt.-%, more preferably 1.5±0.5 wt.-%, still more preferably 1.5±0.4 wt.-%, yet more preferably 1.5±0.3 wt.-%, even more preferably 1.5±0.2 wt.-%, and most preferably 1.5±0.1 wt.-%, in each case based on the total weight of the pharmaceutical dosage form.

Preferably, the relative weight ratio of the ephedrine component to the antioxidant, preferably α-tocopherol, is within the range of from 5:1 to 35:1 or is within the range of from 7:1 to 33:1, more preferably from 9:1 to 31:1, still more preferably from 11:1 to 29:1, yet more preferably from 13:1 to 27:1, even more preferably from 15:1 to 25:1, most preferably from 17:1 to 23:1, and in particular from 19:1 to 21:1.

It has been surprisingly found that comparatively high weight contents of antioxidant, especially of α-tocopherol, provide advantages. It has been found that pharmaceutical dosage forms comprising a higher content of antioxidant, especially α-tocopherol, suppress chemical conversion of the ephedrine or pseudoephedrine into methamphetamine. Furthermore, when trying to extract ephedrine or pseudoephedrine from the dosage forms by means or methylene chloride, the presence of antioxidant, especially α-tocopherol, reduces the extractable amount of ephedrine and pseudoephedrine, respectively.

Preferably, the pharmaceutical dosage form according to the invention comprises a cellulose ether, preferably hydroxypropylmethyl cellulose. The cellulose ether, preferably hydroxypropylmethyl cellulose, is distinct from the cross-linked polymer, preferably croscarmellose or croscarmellose sodium, and the binder, preferably polyvinylpyrrolidone, that may optionally be also contained in the pharmaceutical dosage form according to the invention. Preferably, the cellulose ether, preferably hydroxypropylmethyl cellulose, is substantially linear, i.e. not cross-linked. Preferably, the cellulose ether, preferably hydroxypropylmethyl cellulose, is substantially nonionic, i.e. neither cationic nor anionic. In a preferred embodiment, the pharmaceutical dosage form according to the invention comprises croscarmellose, which from a chemical point of view can also be regarded as a cellulose ether. However, as croscarmellose is cross-linked, for the purpose of the specification croscarmellose is a “cross-linked polymer” (see below), not a “cellulose ether”.

Preferably, the cellulose ether is selected from the group consisting of methyl cellulose, ethyl cellulose, propyl cellulose, hydroxyethyl cellulose, hydroxypropyl cellulose (HPC), hydroxypropylmethyl cellulose (HPMC), carboxymethyl cellulose, salts of carboxymethyl cellulose, and mixtures of any of the foregoing. Hydroxypropylmethyl cellulose is particularly preferred.

Preferably, the weight content of the cellulose ether, preferably hydroxypropylmethyl cellulose, is within the range of from 0.5 to 20 wt.-% or is within the range of from 1.0 to 15 wt.-%, relative to the total weight of the pharmaceutical dosage form.

Preferably, the weight content of the cellulose ether, preferably hydroxypropylmethyl cellulose, is within the range of 7.0±6.0 wt.-%, more preferably 7.0±5.0 wt.-%, still more preferably 7.0±4.0 wt.-%, yet more preferably 7.0±3.0 wt.-%, even more preferably 7.0±2.0 wt.-%, and most preferably 7.0±1.0 wt.-%, in each case based on the total weight of the pharmaceutical dosage form.

In another preferred embodiment, the pharmaceutical dosage form according to the invention does not contain such cellulose ether, preferably hydroxypropylmethyl cellulose.

Preferably, the relative weight ratio of the ephedrine component to the cellulose ether, preferably hydroxypropylmethyl cellulose, preferably hydroxypropylmethyl cellulose, is within the range of from 1:1 to 7.5:1, more preferably from 1.5:1 to 7:1, still more preferably from 2:1 to 6.5:1, yet more preferably from 2.5:1 to 6:1, even more preferably from 3:1 to 5.5:1, most preferably from 3.5:1 to 5:1, and in particular from 4:1 to 4.5:1.

In a preferred embodiment, the relative weight ratio of the polyalkylene oxide to the cellulose ether, preferably hydroxypropylmethyl cellulose, is within the range of from 2.0:1 to 12:1, more preferably 3.0:1 to 11:1, still more preferably 3.5:1 to 10:1, yet more preferably 4.0:1 to 9.5:1, most preferably 4.5:1 to 8.0:1 and in particular 5.0:1 to 7.5:1.

When the pharmaceutical dosage form according to the invention also contains a cross-linked polymer, preferably croscarmellose or croscarmellose sodium, the relative weight ratio of the cross-linked polymer, preferably croscarmellose or croscarmellose sodium, to the cellulose ether, preferably hydroxypropylmethyl cellulose, is preferably within the range of from 4:1 to 1:4, more preferably 3.5:1 to 1:3.5, still more preferably 3:1 to 1:3, yet more preferably 2.5:1 to 1:2.5, most preferably 2:1 to 1:2 and in particular 1.5:1 to 1:1.5.

When the pharmaceutical dosage form according to the invention also contains a binder, preferably polyvinylpyrrolidone, the relative weight ratio of the binder, preferably polyvinylpyrrolidone, to the cellulose ether, preferably hydroxypropylmethyl cellulose, is preferably within the range of from 4:1 to 1:4, more preferably 3.5:1 to 1:3.5, still more preferably 3:1 to 1:3, yet more preferably 2.5:1 to 1:2.5, most preferably 2:1 to 1:2 and in particular 1.5:1 to 1:1.5.

In a preferred embodiment, the cellulose ether, preferably hydroxypropylmethyl cellulose, is homogeneously distributed in the pharmaceutical dosage form according to the invention. Preferably, the ephedrine component and the cellulose ether, preferably hydroxypropylmethyl cellulose, are intimately homogeneously distributed in the pharmaceutical dosage form so that the pharmaceutical dosage form does not contain any segments where either ephedrine component is present in the absence of cellulose ether, preferably hydroxypropylmethyl cellulose, or where cellulose ether, preferably hydroxypropylmethyl cellulose, is present in the absence of ephedrine component.

When the pharmaceutical dosage form is film coated, the cellulose ether, preferably hydroxypropylmethyl cellulose, is preferably homogeneously distributed in the core of the pharmaceutical dosage form. The film coating as such may also contain one or more polymers including cellulose ethers, which may differ from the cellulose ether, preferably hydroxypropylmethyl cellulose, contained in the core or may be identical.

Preferably, the pharmaceutical dosage form according to the invention comprises a cross-linked polymer, preferably croscarmellose or croscarmellose sodium. The cross-linked polymer, preferably croscarmellose or croscarmellose sodium, is distinct from the cellulose ether, preferably hydroxypropylmethyl cellulose, and the binder, preferably polyvinylpyrrolidone, that may optionally be also contained in the pharmaceutical dosage form according to the invention. Cross-linked cellulose ethers are preferably regarded as “cross-linked polymers”, but not as “cellulose ethers” according to the invention.

Preferably, the cross-linked polymer is selected from the group consisting of croscarmellose, salts or croscarmellose, crospovidone, and mixtures of any of the foregoing.

Preferably, the weight content of the cross-linked polymer, preferably croscarmellose or croscarmellose sodium, is within the range of from 1.0 to 15 wt.-%, relative to the total weight of the pharmaceutical dosage form.

When the cross-linked polymer is anionic, e.g. croscarmellose, preferably at least some of the anionic functional groups, e.g. carboxylate and/or sulfonate anions, contained in the anionic cross-linked polymer, preferably croscarmellose or croscarmellose sodium, are present in neutralized form, i.e. they are not present in their protonated forms, but are salts with salt-forming cations instead. Suitable salt-forming cations include alkali metal, ammonium, substituted ammonium and amines. More preferably, at least some of the anionic functional groups, e.g. carboxylate and/or sulfonate anions, are salts of sodium or potassium cations.

In a particularly preferred embodiment, the cross-linked polymer is croscarmellose or a physiologically acceptable salt thereof. Preferably, the cross-linked polymer is croscarmellose sodium. Preferably, the croscarmellose sodium is in accordance with monograph E-09 Croscarmellose Sodium of USP, preferably in the version of 2016.

Croscarmellose sodium is an internally cross-linked sodium carboxymethylcellulose typically used as a superdisintegrant in pharmaceutical formulations. The cross-linking reduces water solubility while still allowing the material to swell and absorb many times its weight in water. Its purpose in most tablets—including dietary supplements—is to assist the tablet in disintegrating in the gastrointestinal tract promptly. Croscarmellose can be made by first soaking crude cellulose in sodium hydroxide, and then reacting the cellulose with sodium monochloroacetate to form sodium carboxymethylcellulose. Excess sodium monochloroacetate slowly hydrolyzes to glycolic acid and the glycolic acid catalyzes the cross-linkage to form croscarmellose sodium. Chemically, croscarmellose sodium is the sodium salt of a cross-linked, partly O-(carboxymethylated) cellulose.

The weight content of the cross-linked polymer, preferably croscarmellose or croscarmellose sodium, is preferably within the range of from 1.0 to 35 wt.-%, more preferably from 5.0 to 35 wt.-%, still more preferably from 1.0 to 15 wt.-%, based on the total weight of the pharmaceutical dosage form.

Preferably, the weight content of the cross-linked polymer, preferably croscarmellose or croscarmellose sodium, is within the range of 7.0±6.0 wt.-%, more preferably 7.0±5.0 wt.-%, still more preferably 7.0±4.0 wt.-%, yet more preferably 7.0±3.0 wt.-%, even more preferably 7.0±2.0 wt.-%, and most preferably 7.0±1.0 wt.-%, in each case based on the total weight of the pharmaceutical dosage form.

A comparison of cross-linked croscarmellose with linear hydroxypropylmethyl cellulose (cellulose ether according to the invention) revealed that under otherwise identical conditions, the cross-linked croscarmellose provided better resistance against solvent extraction than the linear hydroxypropylmethyl cellulose with and without addition of sodium hydroxide in methylene chloride, diethylether and ethyl acetate.

In another preferred embodiment, the pharmaceutical dosage form according to the invention does not contain such cross-linked polymer, preferably croscarmellose or croscarmellose sodium.

In a preferred embodiment, the relative weight ratio of the polyalkylene oxide to the cross-linked polymer, preferably croscarmellose or croscarmellose sodium, is within the range of from 2.0:1 to 12:1, more preferably 3.0:1 to 11:1, still more preferably 3.5:1 to 10:1, yet more preferably 4.0:1 to 9.5:1, most preferably 4.5:1 to 8.0:1 and in particular 5.0:1 to 7.5:1.

When the pharmaceutical dosage form according to the invention also contains a cellulose ether, preferably hydroxypropylmethyl cellulose, the relative weight ratio of the cellulose ether, preferably hydroxypropylmethyl cellulose, to the cross-linked polymer, preferably croscarmellose or croscarmellose sodium, is preferably within the range of from 4:1 to 1:4, more preferably 3.5:1 to 1:3.5, still more preferably 3:1 to 1:3, yet more preferably 2.5:1 to 1:2.5, most preferably 2:1 to 1:2 and in particular 1.5:1 to 1:1.5.

When the pharmaceutical dosage form according to the invention also contains a binder, preferably polyvinylpyrrolidone, the relative weight ratio of the binder, preferably polyvinylpyrrolidone, to the cross-linked polymer, preferably croscarmellose or croscarmellose sodium, is preferably within the range of from 4:1 to 1:4, more preferably 3.5:1 to 1:3.5, still more preferably 3:1 to 1:3, yet more preferably 2.5:1 to 1:2.5, most preferably 2:1 to 1:2 and in particular 1.5:1 to 1:1.5.

In a preferred embodiment, the cross-linked polymer, preferably croscarmellose or croscarmellose sodium, is homogeneously distributed in the pharmaceutical dosage form according to the invention. Preferably, the ephedrine component and the cross-linked polymer, preferably croscarmellose or croscarmellose sodium, are intimately homogeneously distributed in the pharmaceutical dosage form so that the pharmaceutical dosage form does not contain any segments where either ephedrine component is present in the absence of cross-linked polymer or where cross-linked polymer is present in the absence of ephedrine component.

When the pharmaceutical dosage form is film coated, the cross-linked polymer, preferably croscarmellose or croscarmellose sodium, is preferably homogeneously distributed in the core of the pharmaceutical dosage form, i.e. the film coating preferably does not contain cross-linked polymer. Nonetheless, the film coating as such may of course contain one or more polymers, which however, preferably differ from the cross-linked polymer contained in the core.

Preferably, the pharmaceutical dosage form according to the invention comprises a binder, preferably polyvinylpyrrolidone. The binder, preferably polyvinylpyrrolidone, is distinct from the cellulose ether, preferably hydroxypropylmethyl cellulose, and the cross-linked polymer, preferably croscarmellose or croscarmellose sodium, that may optionally be also contained in the pharmaceutical dosage form according to the invention.

Preferably, the binder is selected from the group consisting of disaccharides, starch, modified starch, sugar alcohols, polyvinylpyrrolidone, and mixtures of any of the foregoing. Polyvinylpyrrolidone is particularly preferred.

Preferably, the weight content of the binder, preferably polyvinylpyrrolidone, is within the range of from 1.0 to 15 wt.-%, relative to the total weight of the pharmaceutical dosage form.

Preferably, the weight content of the binder, preferably polyvinylpyrrolidone, is within the range of 7.0±6.0 wt.-%, more preferably 7.0±5.0 wt.-%, still more preferably 7.0±4.0 wt.-%, yet more preferably 7.0±3.0 wt.-%, even more preferably 7.0±2.0 wt.-%, and most preferably 7.0±1.0 wt.-%, in each case based on the total weight of the pharmaceutical dosage form.

Preferably, the relative weight ratio of the ephedrine component to the binder, preferably polyvinylpyrrolidone, is within the range of from 1:1 to 7.5:1, more preferably from 1.5:1 to 7:1, still more preferably from 2:1 to 6.5:1, yet more preferably from 2.5:1 to 6:1, even more preferably from 3:1 to 5.5:1, most preferably from 3.5:1 to 5:1, and in particular from 4:1 to 4.5:1.

In another preferred embodiment, the pharmaceutical dosage form according to the invention does not contain such binder.

In a preferred embodiment, the relative weight ratio of the polyalkylene oxide to the binder, preferably polyvinylpyrrolidone, is within the range of from 2.0:1 to 12:1, more preferably 3.0:1 to 11:1, still more preferably 3.5:1 to 10:1, yet more preferably 4.0:1 to 9.5:1, most preferably 4.5:1 to 8.0:1 and in particular 5.0:1 to 7.5:1.

When the pharmaceutical dosage form according to the invention also contains a cellulose ether, preferably hydroxypropylmethyl cellulose, the relative weight ratio of the cellulose ether, preferably hydroxypropylmethyl cellulose, to the binder, preferably polyvinylpyrrolidone, is preferably within the range of from 4:1 to 1:4, more preferably 3.5:1 to 1:3.5, still more preferably 3:1 to 1:3, yet more preferably 2.5:1 to 1:2.5, most preferably 2:1 to 1:2 and in particular 1.5:1 to 1:1.5.

When the pharmaceutical dosage form according to the invention also contains a cross-linked polymer, preferably croscarmellose or croscarmellose sodium, the relative weight ratio of the cross-linked polymer, preferably croscarmellose or croscarmellose sodium, to the binder, preferably polyvinylpyrrolidone, is preferably within the range of from 4:1 to 1:4, more preferably 3.5:1 to 1:3.5, still more preferably 3:1 to 1:3, yet more preferably 2.5:1 to 1:2.5, most preferably 2:1 to 1:2 and in particular 1.5:1 to 1:1.5.

In a preferred embodiment, the binder, preferably polyvinylpyrrolidone, is homogeneously distributed in the pharmaceutical dosage form according to the invention. Preferably, the ephedrine component and the binder, preferably polyvinylpyrrolidone, are intimately homogeneously distributed in the pharmaceutical dosage form so that the pharmaceutical dosage form does not contain any segments where either ephedrine component is present in the absence of binder, preferably polyvinylpyrrolidone, or where binder, preferably polyvinylpyrrolidone, is present in the absence of ephedrine component.

When the pharmaceutical dosage form is film coated, the binder, preferably polyvinylpyrrolidone, is preferably homogeneously distributed in the core of the pharmaceutical dosage form, i.e. the film coating preferably does not contain binder, preferably polyvinylpyrrolidone. Nonetheless, the film coating as such may of course contain one or more polymers, which however, preferably differ from the binder, preferably polyvinylpyrrolidone, contained in the core.

In a preferred embodiment, the pharmaceutical dosage form according to the invention comprises a cellulose ether, preferably hydroxypropylmethyl cellulose, as defined above, but preferably neither a cross-linked polymer, preferably croscarmellose or croscarmellose sodium, as defined above nor a binder as defined above.

In another preferred embodiment, the pharmaceutical dosage form according to the invention comprises a cross-linked polymer, preferably croscarmellose or croscarmellose sodium, as defined above, but preferably neither a cellulose ether, preferably hydroxypropylmethyl cellulose, as defined above nor a binder as defined above.

In still another preferred embodiment, the pharmaceutical dosage form according to the invention comprises a binder, preferably polyvinylpyrrolidone, as defined above, but preferably neither a cellulose ether, preferably hydroxypropylmethyl cellulose, as defined above nor a cross-linked polymer, preferably croscarmellose or croscarmellose sodium, as defined above.

In yet another preferred embodiment, the pharmaceutical dosage form according to the invention comprises a combination of a cellulose ether, preferably hydroxypropylmethyl cellulose, as defined above with a cross-linked polymer, preferably croscarmellose or croscarmellose sodium, as defined above, but preferably no binder as defined above.

In another preferred embodiment, the pharmaceutical dosage form according to the invention comprises a combination of a cellulose ether as defined above, preferably hydroxypropylmethyl cellulose, with a binder, preferably polyvinylpyrrolidone, as defined above, preferably polyvinylpyrrolidone, but preferably no cross-linked polymer as defined above. Preferably, the pharmaceutical dosage form additionally comprises an elevated quantity of antioxidant, preferably of α-tocopherol. It has been surprisingly found that under standardized conditions in comparison to other formulations, this particularly preferred pharmaceutical dosage form according to the invention provides
coarse particles upon grinding of under harsh conditions (88 wt.-% of material with particle size >1 mm, whereas other formulations only provided at most 55 wt.-% of material with particle size >1 mm);
a low quantity of methamphetamine by chemical conversion;
a low quantity of pseudoephedrine that could be extracted in Vodka; and
a low quantity of pseudoephedrine that could be extracted with diethylether or ethylacetate, with our without prior treatment with sodium hydroxide.

In still another preferred embodiment, the pharmaceutical dosage form according to the invention comprises a combination of a cross-linked polymer as defined above, preferably croscarmellose or croscarmellose sodium, with a binder, preferably polyvinylpyrrolidone, as defined above, preferably polyvinylpyrrolidone, but preferably no cellulose ether, preferably hydroxypropylmethyl cellulose, as defined above. Preferably, the pharmaceutical dosage form additionally comprises an elevated quantity of antioxidant, preferably of α-tocopherol. It has been surprisingly found that under standardized conditions in comparison to other formulations, this particularly preferred pharmaceutical dosage form according to the invention provides
coarse particles upon grinding of under harsh conditions (88 wt.-% of material with particle size >1 mm, whereas other formulations only provided at most 55 wt.-% of material with particle size >1 mm);
a low quantity of methamphetamine by chemical conversion;
a low quantity of pseudoephedrine that could be extracted in Vodka; and
a low quantity of pseudoephedrine that could be extracted with diethylether or ethylacetate, with our without prior treatment with sodium hydroxide.

In a further preferred embodiment, the pharmaceutical dosage form according to the invention comprises a combination of a cellulose ether, preferably hydroxypropylmethyl cellulose, as defined above with a cross-linked polymer, preferably croscarmellose or croscarmellose sodium, as defined above and with a binder, preferably polyvinylpyrrolidone, as defined above.

Preferably, the pharmaceutical dosage form according to the invention comprises a binder as defined above, preferably polyvinylpyrrolidone, in combination with
either a cross-linked polymer as defined above, preferably croscarmellose or croscarmellose sodium;
or a cellulose ether as defined above, preferably hydroxypropylmethyl cellulose;
   and in either case preferably additionally an elevated quantity of antioxidant, preferably of α-tocopherol.

It has been surprisingly found that polyvinylpyrrolidone (PVP) and croscarmellose, particularly when being combined with one another, provide pharmaceutical dosage forms that yield coarse particles upon grinding, if the pharmaceutical dosage forms can be ground at all. Yet further, when trying to extract the ephedrine or pseudoephedrine from such coarse particles with water and diethylether, a stabile emulsion is formed, i.e. the ether phase does not separate from the water phase so that extraction by means of a separatory funnel is not at all or at least hardly possible.

Further, there is indication that polyvinylpyrrolidone (PVP) exhibits a certain solubility in those solvents that are conventionally used by abusers in attempts of extracting ephedrine or pseudoephedrine from pharmaceutical dosage forms or attempts of chemically converting ephedrine or pseudoephedrine to methamphetamine. This results in a desirable impurity of the obtained intermediate extract or yielded product thus contaminating the intermediate extract or final product and impeding further abuse and administration thereof, respectively.

In a preferred embodiment, the pharmaceutical dosage form according to the invention additionally comprises a plasticizer preferably selected from the group consisting of polyalkylene glycol, triacetin, fatty acids, fatty acid esters, waxes and microcrystalline waxes. Particularly preferred plasticizers are polyethylene glycols, such as PEG 6000.

Preferably, the weight content of the plasticizer is preferably within the range of 10±9 wt.-%, based on the total weight of the pharmaceutical dosage form. Preferably, the weight content of the plasticizer is within the range of 10±8 wt.-%, more preferably 10±7 wt.-%, still more preferably 10±6 wt.-%, yet more preferably 10±5 wt.-%, even more preferably 10±4 wt.-%, and most preferably 10±3 wt.-%, based on the total weight of the pharmaceutical dosage form.

Besides the ephedrine component, the optionally present polyalkylene oxide, the optionally present antioxidant, the optionally present plasticizer, the optionally present cellulose ether, preferably hydroxypropylmethyl cellulose, the optionally present cross-linked polymer, preferably croscarmellose or croscarmellose sodium, and the optionally present binder, preferably polyvinylpyrrolidone, the pharmaceutical dosage form according to the invention may contain further ingredients, e.g. one or more conventional pharmaceutical excipient(s), e.g. fillers, glidants, granulating agents, anti-caking agents, lubricants, flavors, dyes, and/or preservatives.

The pharmaceutical dosage form according to the invention is preferably an oral pharmaceutical dosage form, particularly a tablet.

It is also possible, however, to administer the pharmaceutical dosage form via different routes and thus, the pharmaceutical dosage form may alternatively be adapted for buccal, lingual, rectal or vaginal administration. Implants are also possible.

In a preferred embodiment, the pharmaceutical dosage form is multiparticulate, preferably a capsule. Under these circumstances, not the capsule as such, but at least a portion of the particles that are contained in the capsule have a breaking strength of at least 300 N.

In another preferred embodiment, the pharmaceutical dosage form is monolithic. Preferably, the pharmaceutical dosage form is neither in film form, nor multi-particulate.

In a preferred embodiment, the pharmaceutical dosage form according to the invention is a round tablet. Tablets of this embodiment preferably have a diameter in the range of about 1 mm to about 30 mm, in particular in the range of about 2 mm to about 25 mm, more in particular about 5 mm to about 23 mm, even more in particular about 7 mm to about 13 mm; and a thickness in the range of about 1.0 mm to about 12 mm, in particular in the range of about 2.0 mm to about 10 mm, even more in particular from 3.0 mm to about 9.0 mm, even further in particular from about 4.0 mm to about 8.0 mm.

In another preferred embodiment, the pharmaceutical dosage form according to the invention is an oblong tablet. Tablets of this embodiment preferably have a lengthwise extension (longitudinal extension) of about 1 mm to about 30 mm, in particular in the range of about 2 mm to about 25 mm, more in particular about 5 mm to about 23 mm, even more in particular about 7 mm to about 20 mm; and a thickness in the range of about 1.0 mm to about 12 mm, in particular in the range of about 2.0 mm to about 10 mm, even more in particular from 3.0 mm to about 9.0 mm, even further in particular from about 4.0 mm to about 8.0 mm.

The pharmaceutical dosage form according to the invention has preferably a weight in the range of 0.01 to 1.5 g, more preferably in the range of 0.05 to 1.2 g, still more preferably in the range of 0.1 g to 1.0 g, yet more preferably in the range of 0.2 g to 0.9 g, and most preferably in the range of 0.25 g to 0.8 g.

The pharmaceutical dosage form of the invention can optionally be provided, partially or completely, with a conventional coating. The pharmaceutical dosage forms of the present invention are preferably film coated with conventional film coating compositions.

Suitable coating materials are commercially available, e.g. under the trademarks Opadry® and Eudragit®.

Examples of suitable materials include cellulose esters and cellulose ethers, such as methylcellulose (MC), hydroxypropylmethyl cellulose (HPMC), hydroxypropylcellulose (HPC), hydroxyethylcellulose (HEC), sodium carboxymethylcellulose (Na-CMC), ethylcellulose (EC), cellulose acetate phthalate (CAP), hydroxypropylmethyl cellulose phthalate (HPMCP); poly(meth)acrylates, such as aminoalkylmethacrylate copolymers, ethylacrylate methyl-methacrylate copolymers, methacrylic acid methylmethacrylate copolymers, methacrylic acid methylmethacrylate copolymers; vinyl polymers, such as polyvinylpyrrolidone, polyvinylacetate-phthalate, polyvinyl alcohol, polyvinylacetate; and natural film formers, such as shellack.

In a particularly preferred embodiment, the coating is water-soluble. In a preferred embodiment, the coating is based on polyvinyl alcohol, such as polyvinyl alcohol-part. hydrolyzed, and may additionally contain polyethylene glycol, such as macrogol 3350, and/or pigments. In another preferred embodiment, the coating is based on hydroxypropylmethyl cellulose, preferably hypromellose type 2910 having a viscosity of 3 to 15 mPa·s.

The coating can be resistant to gastric juices and dissolve as a function of the pH value of the release environment. By means of this coating, it is possible to ensure that the pharmaceutical dosage form according to the invention passes through the stomach undissolved and the active ingredient is only released in the intestines. The coating which is resistant to gastric juices preferably dissolves at a pH value of between 5 and 7.5. Corresponding materials and methods for the delayed release of active ingredients and for the application of coatings which are resistant to gastric juices are known to the person skilled in the art, for example from “Coated Pharmaceutical dosage forms—Fundamentals, Manufacturing Techniques, Biopharmaceutical Aspects, Test Methods and Raw Materials” by Kurt H. Bauer, K. Lehmann, Hermann P. Osterwald, Rothgang, Gerhart, 1st edition, 1998, Medpharm Scientific Publishers.

The coating can also be applied e.g. to improve the aesthetic impression and/or the taste of the pharmaceutical dosage forms and the ease with which they can be swallowed. Coating the pharmaceutical dosage forms of the present invention can also serve other purposes, e.g. improving stability and shelf-life. Suitable coating formulations comprise a film forming polymer such as, for example, polyvinyl alcohol or hydroxypropylmethyl cellulose, e.g. hypromellose, a plasticizer such as, for example, a glycol, e.g. propylene glycol or polyethylene glycol, an opacifier, such as, for example, titanium dioxide, and a film smoothener, such as, for example, talc. Suitable coating solvents are water as well as organic solvents. Examples of organic solvents are alcohols, e.g. ethanol or isopropanol, ketones, e.g. acetone, or halogenated hydrocarbons, e.g. methylene chloride. Optionally, the coating can contain a therapeutically effective amount of one or more active ingredients to provide for an immediate release of said ephedrine component and thus for an immediate relief of the symptoms treated by said ephedrine component. Coated pharmaceutical dosage forms of the present invention are preferably prepared by first making the cores and subsequently coating said cores using conventional techniques, such as coating in a coating pan.

According to the invention, the ephedrine component is preferably embedded in a controlled-release matrix comprising a polyalkylene oxide.

Controlled release of an active ingredient from an oral pharmaceutical dosage form is known to a person skilled in the art. For the purpose of the specification, controlled release encompasses delayed release, retarded release, sustained release, prolonged release, and the like.

Controlled or prolonged release is understood according to the invention preferably to mean a release profile in which the ephedrine component is released over a relatively long period with reduced intake frequency with the purpose of extended therapeutic action. Preferably, the meaning of the term “prolonged release” is in accordance with the European guideline on the nomenclature of the release profile of pharmaceutical dosage forms (CHMP). This is achieved in particular with peroral administration. The expression “at least partially delayed or prolonged release” covers according to the invention any pharmaceutical dosage forms which ensure modified release of the opioids (A) contained therein. The pharmaceutical dosage forms preferably comprise coated or uncoated pharmaceutical dosage forms, which are produced with specific auxiliary substances, by particular processes or by a combination of the two possible options in order purposefully to change the release rate or location of release.

In the case of the pharmaceutical dosage forms according to the invention, the release time profile of a controlled release form may be modified e.g. as follows: extended release, repeat action release, prolonged release and sustained release.

For the purpose of the specification “controlled release” preferably means a product in which the release of active ingredient over time is controlled by the type and composition of the formulation. For the purpose of the specification “extended release” preferably means a product in which the release of active ingredient is delayed for a finite lag time, after which release is unhindered. For the purpose of the specification “repeat action release” preferably means a product in which a first portion of active ingredient is released initially, followed by at least one further portion of active ingredient being released subsequently. For the purpose of the specification “prolonged release” preferably means a product in which the rate of release of active ingredient from the formulation after administration has been reduced over time, in order to maintain therapeutic activity, to reduce toxic effects, or for some other therapeutic purpose. For the purpose of the specification “sustained release” preferably means a way of formulating a medicine so that it is released into the body steadily, over a long period of time, thus reducing the dosing frequency. For further details, reference may be made, for example, to K. H. Bauer, Lehrbuch der Pharmazeutischen Technologie, 6th edition, WVG Stuttgart, 1999; and Eur. Ph.

Preferably the pharmaceutically pharmaceutical dosage form provides a release of the ephedrine component after 1 hour of preferably at most 60%, more preferably at most 40%, yet more preferably at most 30%, still more preferably at most 20% and most preferably at most 17%. After 2 hour preferably at most 80%, more preferably at most 60%, yet more preferably at most 50%, still more preferably at most 40% and most preferably at most 32%. After 3 hour preferably at most 85%, more preferably at most 65%, yet more preferably at most 55%, still more preferably at most 48% and most preferably at most 42%. After 4 hour preferably at most 90%, more preferably at most 75%, yet more preferably at most 65%, still more preferably at most 55% and most preferably at most 49%. After 7 hour preferably at most 95%, more preferably at most 85%, yet more preferably at most 80%, still more preferably at most 70% and most preferably at most 68%. After 10 hour preferably at most 99%, more preferably at most 90%, yet more preferably at most 88%, still more preferably at most 83% and most preferably at most 80%. After 13 hour preferably at most 99%, more preferably at most 95%, yet more preferably at most 93%, still more preferably at most 91% and most preferably at most 89%.

In a preferred embodiment, the pharmaceutical dosage form according to the invention which has released under in vitro conditions:

after 1 h at most 40 wt.-%,

after 2 h at most 55 wt.-%,

after 3 h at most 70 wt.-%, and

after 4 h at most 85 wt.-%

of the total content of the ephedrine component that was originally contained in the pharmaceutical dosage form.

In another preferred embodiment, the pharmaceutical dosage form according to the invention provides immediate release of the ephedrine component. For the purpose of the specification, immediate release preferably means that under in vitro conditions after 30 minutes at least 80 wt.-% of the ephedrine component have been released which was originally contained in the pharmaceutical dosage form.

Immediate release is preferably achieved by means of a multiparticulate pharmaceutical dosage form, preferably in form of a capsule, or a MUPS formulation, wherein at least a portion of the individual particles that contain the ephedrine, preferably all such particles, have a breaking strength of at least 300 N.

Break resistant multiparticulate pharmaceutical dosage forms providing immediate release of pharmacologically active ingredients are known from the prior art, e.g. from WO 2008/107149, US 2013 0028970 and US 2013 0028972, which are incorporated by reference.

Suitable in vitro conditions are known to the skilled artisan. In this regard it can be referred to, e.g., the Eur. Ph. Preferably, the release profile is measured under the following conditions: Paddle apparatus equipped with sinker, 75 rpm, 37±5° C., 900 mL simulated intestinal fluid pH 6.8 (phosphate buffer) or pH 4.5. In a preferred embodiment, the rotational speed of the paddle is increased to 100 rpm.

Preferably, the pharmaceutical dosage form for use according to the invention is administered once daily, twice daily or thrice daily. Preferably, the pharmaceutical dosage form according to the invention is for use in therapy, wherein the pharmaceutical dosage form is administered once daily, or twice daily, or thrice daily. Thus, in a preferred embodiment, the pharmaceutical dosage form according to the invention is adapted for administration once daily. In another preferred embodiment, the pharmaceutical dosage form according to the invention is adapted for administration twice daily. In still another preferred embodiment, the pharmaceutical dosage form according to the invention is adapted for administration thrice daily.

For the purpose of the specification, “twice daily” means equal or nearly equal time intervals, i.e., about every 12 hours, or different time intervals, e.g., 8 and 16 hours or 10 and 14 hours, between the individual administrations.

For the purpose of the specification, “thrice daily” means equal or nearly equal time intervals, i.e., about every 8 hours, or different time intervals, e.g., 6, 6 and 12 hours; or 7, 7 and 10 hours, between the individual administrations.

The pharmaceutical dosage form according to the invention has a breaking strength of at least 300 N, preferably at least 500 N. When the pharmaceutical dosage form is monolithic, e.g. a tablet, the monolithic pharmaceutical dosage form as such has a breaking strength of at least 300 N. When the pharmaceutical dosage form is multiparticulate, e.g. a capsule, preferably not the capsule material but at least a portion of the particles that are contained in the capsule have a breaking strength of at least 300 N.

The pharmaceutical dosage form according to the invention is preferably tamper-resistant. Preferably, tamper-resistance is achieved based on the mechanical properties of the pharmaceutical dosage form so that comminution is avoided or at least substantially impeded. According to the invention, the term comminution means the pulverization of the pharmaceutical dosage form using conventional means usually available to an abuser, for example a pestle and mortar, a hammer, a mallet or other conventional means for pulverizing under the action of force. Thus, tamper-resistance preferably means that pulverization of the pharmaceutical dosage form using conventional means is avoided or at least substantially impeded.

Preferably, the mechanical properties of the pharmaceutical dosage form according to the invention, particularly its breaking strength, substantially rely on the presence and spatial distribution of polyalkylene oxide, although its mere presence does typically not suffice in order to achieve said properties. The advantageous mechanical properties of the pharmaceutical dosage form according to the invention may not automatically be achieved by simply processing ephedrine component, polyalkylene oxide, and optionally further excipients by means of conventional methods for the preparation of pharmaceutical dosage forms. In fact, usually suitable apparatuses must be selected for the preparation and critical processing parameters must be adjusted, particularly pressure/force, temperature and time. Thus, even if conventional apparatuses are used, the process protocols usually must be adapted in order to meet the required criteria.

In general, the pharmaceutical dosage forms exhibiting the desired properties may be obtained only if, during preparation of the pharmaceutical dosage form, suitable components

in suitable amounts

are exposed to

a sufficient pressure

at a sufficient temperature

for a sufficient period of time.

Thus, regardless of the apparatus used, the process protocols must be adapted in order to meet the required criteria. Therefore, the breaking strength is separable from the composition.

The pharmaceutical dosage form according to the invention has a breaking strength of at least 300 N, preferably at least 500 N, preferably at least 600 N, more preferably at least 700 N, still more preferably at least 800 N, yet more preferably at least 1000 N, most preferably at least 1250 N and in particular at least 1500 N.

The “breaking strength” (resistance to crushing) of a pharmaceutical dosage form is known to the skilled person. In this regard it can be referred to, e.g., W. A. Ritschel, Die Tablette, 2. Auflage, Editio Cantor Verlag Aulendorf, 2002; H Liebermann et al., Pharmaceutical dosage forms: Tablets, Vol. 2, Informa Healthcare; 2 edition, 1990; and Encyclopedia of Pharmaceutical Technology, Informa Healthcare; 1 edition.

For the purpose of the specification, the breaking strength is preferably defined as the amount of force that is necessary in order to fracture the pharmaceutical dosage form (=breaking force). Therefore, for the purpose of the specification the pharmaceutical dosage form does preferably not exhibit the desired breaking strength when it breaks, i.e., is fractured into at least two independent parts that are separated from one another. In another preferred embodiment, however, the pharmaceutical dosage form is regarded as being broken if the force decreases by 25% (threshold value) of the highest force measured during the measurement (see below).

The pharmaceutical dosage forms according to the invention are distinguished from conventional pharmaceutical dosage forms in that, due to their breaking strength, they cannot be pulverized by the application of force with conventional means, such as for example a pestle and mortar, a hammer, a mallet or other usual means for pulverization, in particular devices developed for this purpose (tablet crushers). In this regard “pulverization” means crumbling into small particles that would immediately release the ephedrine component in a suitable medium.

Conventional tablets typically have a breaking strength well below 200 N in any direction of extension. The breaking strength of conventional round tablets may be estimated according to the following empirical formula: Breaking Strength [in N]=10×Diameter Of The Tablet [in mm]. Thus, according to said empirical formula, a round tablet having a breaking strength of at least 300 N would require a diameter of at least 30 mm). Such a tablet, however, could not be swallowed. The above empirical formula preferably does not apply to the pharmaceutical dosage forms of the invention, which are not conventional but rather special.

Further, the actual mean chewing force is about 220 N (cf, e.g., P. A. Proeschel et al., J Dent Res, 2002, 81(7), 464-468). This means that conventional tablets having a breaking strength well below 200 N may be crushed upon spontaneous chewing, whereas the pharmaceutical dosage forms according to the invention may not.

Still further, when applying a gravitational acceleration of about 9.81 m/s², 500 N correspond to a gravitational force of more than 50 kg, i.e. the pharmaceutical dosage forms according to the invention can preferably withstand a weight of more than 50 kg without being pulverized.

Methods for measuring the breaking strength of a pharmaceutical dosage form are known to the skilled artisan. Suitable devices are commercially available.

For example, the breaking strength (resistance to crushing) can be measured in accordance with the Eur. Ph. 5.0, 2.9.8 or 6.0, 2.09.08 “Resistance to Crushing of Tablets”. The test is intended to determine, under defined conditions, the resistance to crushing of tablets, measured by the force needed to disrupt them by crushing. The apparatus consists of 2 jaws facing each other, one of which moves towards the other. The flat surfaces of the jaws are perpendicular to the direction of movement. The crushing surfaces of the jaws are flat and larger than the zone of contact with the tablet. The apparatus is calibrated using a system with a precision of 1 Newton. The tablet is placed between the jaws, taking into account, where applicable, the shape, the break-mark and the inscription; for each measurement the tablet is oriented in the same way with respect to the direction of application of the force (and the direction of extension in which the breaking strength is to be measured). The measurement is carried out on 10 tablets, taking care that all fragments of tablets have been removed before each determination. The result is expressed as the mean, minimum and maximum values of the forces measured, all expressed in Newton.

A similar description of the breaking strength (breaking force) can be found in the USP. The breaking strength can alternatively be measured in accordance with the method described therein where it is stated that the breaking strength is the force required to cause a tablet to fail (i.e., break) in a specific plane. The tablets are generally placed between two platens, one of which moves to apply sufficient force to the tablet to cause fracture. For conventional, round (circular cross-section) tablets, loading occurs across their diameter (sometimes referred to as diametral loading), and fracture occurs in the plane. The breaking force of tablets is commonly called hardness in the pharmaceutical literature; however, the use of this term is misleading. In material science, the term hardness refers to the resistance of a surface to penetration or indentation by a small probe. The term crushing strength is also frequently used to describe the resistance of tablets to the application of a compressive load. Although this term describes the true nature of the test more accurately than does hardness, it implies that tablets are actually crushed during the test, which is often not the case.

Alternatively, the breaking strength (resistance to crushing) can be measured in accordance with WO 2005/016313, WO 2005/016314, and WO 2006/082099, which can be regarded as a modification of the method described in the Eur. Ph. The apparatus used for the measurement is preferably a “Zwick Z 2.5” materials tester, Fₘₐₓ=2.5 kN with a maximum draw of 1150 mm, which should be set up with one column and one spindle, a clearance behind of 100 mm and a test speed adjustable between 0.1 and 800 mm/min together with testControl software. Measurement is performed using a pressure piston with screw-in inserts and a cylinder (diameter 10 mm), a force transducer, Fₘₐₓ. 1 kN, diameter=8 mm, class 0.5 from 10 N, class 1 from 2 N to ISO 7500-1, with manufacturer's test certificate M according to DIN 55350-18 (Zwick gross force Fₘₐₓ=1.45 kN) (all apparatus from Zwick GmbH & Co. KG, Ulm, Germany) with Order No BTC-FR 2.5 TH. D09 for the tester, Order No BTC-LC 0050N. P01 for the force transducer, Order No BO 70000 S06 for the centering device.

In a preferred embodiment of the invention, the breaking strength is measured by means of a breaking strength tester e.g. Sotax®, type HT100 or type HT1 (Allschwil, Switzerland). Both, the Sotax® HT100 and the Sotax® HT1 can measure the breaking strength according to two different measurement principles: constant speed (where the test jaw is moved at a constant speed adjustable from 5-200 mm/min) or constant force (where the test jaw increases force linearly adjustable from 5-100 N/sec). In principle, both measurement principles are suitable for measuring the breaking strength of the pharmaceutical dosage form according to the invention. Preferably, the breaking strength is measured at constant speed, preferably at a constant speed of 120 mm/min.

In a preferred embodiment, the pharmaceutical dosage form is regarded as being broken if it is fractured into at least two separate pieces.

The pharmaceutical dosage form according to the invention preferably exhibits mechanical strength over a wide temperature range, in addition to the breaking strength (resistance to crushing) optionally also sufficient hardness, impact resistance, impact elasticity, tensile strength and/or modulus of elasticity, optionally also at low temperatures (e.g. below −24° C., below −40° C. or in liquid nitrogen), for it to be virtually impossible to pulverize by spontaneous chewing, grinding in a mortar, pounding, etc. Thus, preferably, in direction of extension E₁ the comparatively high breaking strength of the pharmaceutical dosage form according to the invention is maintained even at low or very low temperatures, e.g., when the pharmaceutical dosage form is initially chilled to increase its brittleness, for example to temperatures below −25° C., below −40° C. or even in liquid nitrogen.

The pharmaceutical dosage form according to the invention is characterized by a certain degree of breaking strength. This does not mean that the pharmaceutical dosage form must also exhibit a certain degree of hardness. Hardness and breaking strength are different physical properties. Therefore, the tamper resistance of the pharmaceutical dosage form does not necessarily depend on the hardness of the pharmaceutical dosage form. For instance, due to its breaking strength, impact strength, elasticity modulus and tensile strength, respectively, the pharmaceutical dosage form can preferably be deformed, e.g. plastically, when exerting an external force, for example using a hammer, but cannot be pulverized, i.e., crumbled into a high number of fragments. In other words, the pharmaceutical dosage form according to the invention is characterized by a certain degree of breaking strength, but not necessarily also by a certain degree of form stability.

Therefore, in the meaning of the specification, a pharmaceutical dosage form that is deformed when being exposed to a force in a particular direction of extension but that does not break (plastic deformation or plastic flow) is preferably to be regarded as having the desired breaking strength in said direction of extension.

Preferably, the pharmaceutical dosage form according to the invention provides resistance against chemical conversion of the ephedrine component to methamphetamine, preferably under the conditions of the “shake and bake one pot” procedure, preferably under the specific conditions as further detailed in the experimental section, such that the amount of yielded methamphetamine is not more than 20 mole.-%, more preferably not more than 15 mole.-%, still more preferably not more than 10 mole.-%, yet more preferably not more than 5.0 mole.-%, even more preferably not more than 2.5 mole.-%, and most preferably not more than 1.0 mole.-% of the total molar quantity of the ephedrine component that was originally contained in the pharmaceutical dosage form.

In a preferred embodiment, especially when it is attempted to directly chemically convert the ephedrine component into methamphetamine by the “shake and bake one pot” procedure, i.e. without any prior attempts to extract the ephedrine component, the amount of yielded methamphetamine is not more than 2.0 mole.-%, more preferably not more than 1.8 mole.-%, still more preferably not more than 1.6 mole.-%, yet more preferably not more than 1.4 mole.-%, even more preferably not more than 1.2 mole.-%, and most preferably not more than 1.0 mole.-% of the total molar quantity of the ephedrine component that was originally contained in the pharmaceutical dosage form.

Preferably, the pharmaceutical dosage form according to the invention also provides resistance against chemically converting the ephedrine component into methamphetamine after extracting the ephedrine component from the pharmaceutical dosage form by means of aqueous or organic solvents, e.g. diethyl ether, ethyl acetate, methylene chloride, such that the amount of yielded methamphetamine is not more than 20 mole.-%, more preferably not more than 15 mole.-%, still more preferably not more than 10 mole.-%, yet more preferably not more than 5.0 mole.-%, even more preferably not more than 2.5 mole.-%, and most preferably not more than 1.0 mole.-% of the total molar quantity of the ephedrine component that was originally contained in the pharmaceutical dosage form.

In a preferred embodiment, especially when a considerable amount of the ephedrine component could be extracted from the pharmaceutical dosage form, e.g. up to 80 wt.-%, when it is attempted to chemically convert the thus extracted ephedrine component into methamphetamine, due to the presence of coextracted excipients, the amount of yielded methamphetamine is not more than 5.0 mole.-%, more preferably not more than 4.5 mole.-%, still more preferably not more than 4.0 mole.-%, yet more preferably not more than 3.5 mole.-%, even more preferably not more than 3.0 mole.-%, and most preferably not more than 2.5 mole.-% of the total molar quantity of the ephedrine component that was originally contained in the pharmaceutical dosage form.

In preferred embodiments of the pharmaceutical dosage form according to the invention,
the weight content of the ephedrine component is within the range of from 10 to 50 wt.-%, more preferably from 20 to 40 wt.-%, relative to the total weight of the pharmaceutical dosage form; and/or
the pharmaceutical dosage form comprises a polyalkylene oxide, wherein
   the weight content of the polyalkylene oxide is within the range of from 25 to 65 wt.-%, more preferably from 30 to 60 wt.-%, still more preferably from 35 to 55 wt.-%, yet more preferably from 40 to 50 wt.-%, relative to the total weight of the pharmaceutical dosage form; and/or
   the relative weight ratio of the polyalkylene oxide to the ephedrine component is within the range of from 3:1 to 1:2, more preferably 2.5:1 to 1:1.5, still more preferably from 2:1 to 1:1; and/or
   the polyalkylene oxide has a weight average molecular weight of at least 500,000 g/mol, more preferably at least 750,000 g/mol, still more preferably at least 1,000,000 g/mol; and/or
the pharmaceutical dosage form comprises an antioxidant, preferably α-tocopherol, wherein
   the weight content of the antioxidant, preferably α-tocopherol, is at least 0.5 wt.-%, more preferably at least 0.75 wt.-%, still more preferably at least 1.0 wt.-%, yet more preferably at least 1.25 wt.-%, relative to the total weight of the pharmaceutical dosage form; and/or
   the relative weight ratio of the ephedrine component to the antioxidant, preferably α-tocopherol, is within the range of from 35:1 to 5:1, more preferably 30:1 to 10:1, still more preferably 25:1 to 15:1, yet more preferably 21:1 to 19:1; and/or
the pharmaceutical dosage form comprises a cellulose ether, preferably hydroxypropylmethyl cellulose, wherein
   the weight content of the cellulose ether, preferably hydroxypropylmethyl cellulose, is within the range of from 0.5 to 20 wt.-%, more preferably from 1.0 to 15 wt.-%, still more preferably from 2.5 to 12.5 wt.-%, yet more preferably from 5.0 to 10 wt.-%, relative to the total weight of the pharmaceutical dosage form; and/or
   the relative weight ratio of the ephedrine component to the cellulose ether, preferably hydroxypropylmethyl cellulose, is within the range of from 5.5:1 to 3:1, more preferably from 5:1 to 3.5:1, still more preferably from 4.5:1 to 4:1; and/or
the pharmaceutical dosage form comprises a binder, preferably polyvinylpyrrolidone, wherein
   the weight content of the binder, preferably polyvinylpyrrolidone, is within the range of from 0.5 to 20 wt.-%, more preferably from 1.0 to 15 wt.-%, still more preferably from 2.5 to 12.5 wt.-%, yet more preferably from 5.0 to 10 wt.-%, relative to the total weight of the pharmaceutical dosage form; and/or
   the relative weight ratio of the ephedrine component to the binder, preferably polyvinylpyrrolidone, is within the range of from 5.5:1 to 3:1, more preferably from 5:1 to 3.5:1, still more preferably from 4.5:1 to 4:1; and/or
   the relative weight ratio of the cellulose ether, preferably hydroxypropylmethyl cellulose, to the binder, preferably polyvinylpyrrolidone, is within the range of from 2.5:1 to 1:2.5, more preferably from 2:1 to 1:2, still more preferably from 1.5:1 to 1:1.5.

In preferred embodiments of the pharmaceutical dosage form according to the invention,
the weight content of the ephedrine component is within the range of from 10 to 50 wt.-%, more preferably from 20 to 40 wt.-%, relative to the total weight of the pharmaceutical dosage form; and/or
the pharmaceutical dosage form comprises a polyalkylene oxide, wherein
   the weight content of the polyalkylene oxide is within the range of from 25 to 65 wt.-%, more preferably from 30 to 60 wt.-%, still more preferably from 35 to 55 wt.-%, yet more preferably from 40 to 50 wt.-%, relative to the total weight of the pharmaceutical dosage form; and/or
   the relative weight ratio of the polyalkylene oxide to the ephedrine component is within the range of from 3:1 to 1:2, more preferably 2.5:1 to 1:1.5, still more preferably from 2:1 to 1:1; and/or
   the polyalkylene oxide has a weight average molecular weight of at least 500,000 g/mol, more preferably at least 750,000 g/mol, still more preferably at least 1,000,000 g/mol; and/or
the pharmaceutical dosage form comprises an antioxidant, preferably α-tocopherol, wherein
   the weight content of the antioxidant, preferably α-tocopherol, is at least 0.5 wt.-%, more preferably at least 0.75 wt.-%, still more preferably at least 1.0 wt.-%, yet more preferably at least 1.25 wt.-%, relative to the total weight of the pharmaceutical dosage form; and/or
   the relative weight ratio of the ephedrine component to the antioxidant, preferably α-tocopherol, is within the range of from 35:1 to 5:1, more preferably 30:1 to 10:1, still more preferably 25:1 to 15:1, yet more preferably 21:1 to 19:1; and/or
the pharmaceutical dosage form comprises a cross-linked polymer, preferably croscarmellose or croscarmellose sodium, wherein
   the weight content of the cross-linked polymer, preferably croscarmellose or croscarmellose sodium, is within the range of from 0.5 to 20 wt.-%, more preferably from 1.0 to 15 wt.-%, still more preferably from 2.5 to 12.5 wt.-%, yet more preferably from 5.0 to 10 wt.-%, relative to the total weight of the pharmaceutical dosage form; and/or
   the relative weight ratio of the ephedrine component to the cross-linked polymer, preferably croscarmellose or croscarmellose sodium, is within the range of from 5.5:1 to 3:1, more preferably from 5:1 to 3.5:1, still more preferably from 4.5:1 to 4:1; and/or
the pharmaceutical dosage form comprises a binder, preferably polyvinylpyrrolidone, wherein
   the weight content of the binder, preferably polyvinylpyrrolidone, is within the range of from 0.5 to 20 wt.-%, more preferably from 1.0 to 15 wt.-%, still more preferably from 2.5 to 12.5 wt.-%, yet more preferably from 5.0 to 10 wt.-%, relative to the total weight of the pharmaceutical dosage form; and/or
   the relative weight ratio of the ephedrine component to the binder, preferably polyvinylpyrrolidone, is within the range of from 5.5:1 to 3:1, more preferably from 5:1 to 3.5:1, still more preferably from 4.5:1 to 4:1; and/or
   the relative weight ratio of the cross-linked polymer, preferably croscarmellose or croscarmellose sodium, to the binder, preferably polyvinylpyrrolidone, is within the range of from 2.5:1 to 1:2.5, more preferably from 2:1 to 1:2, still more preferably from 1.5:1 to 1:1.5.

Particularly preferred compositions of the pharmaceutical dosage form according to the invention are compiled as embodiments A¹ to A⁴⁸ in the tables here below (according to these embodiment, the pharmaceutical dosage form according to the invention comprises the specified ingredients in the specified quantities but may additionally comprise further ingredients):

| Ingredient [wt.-%] | A¹ | A² | A³ | A⁴ | A⁵ | A⁶ | A⁷ | A⁸ |
|---|---|---|---|---|---|---|---|---|
| ephedrine component | 30 ± 25 | 30 ± 25 | 30 ± 25 | 30 ± 25 | 30 ± 25 | 30 ± 25 | 30 ± 25 | 30 ± 25 |
| polyalkylene oxide | 45 ± 25 | 45 ± 25 | 45 ± 25 | 45 ± 25 | 45 ± 25 | 45 ± 25 | 45 ± 25 | 45 ± 25 |
| plasticizer | 10 ± 9.0 | 10 ± 9.0 | 10 ± 9.0 | 10 ± 9.0 | 10 ± 9.0 | 10 ± 9.0 | 10 ± 9.0 | 10 ± 9.0 |
| antioxidant | 1.5 ± 1.4 | 1.5 ± 1.4 | 1.5 ± 1.4 | 1.5 ± 1.4 | 1.5 ± 1.4 | 1.5 ± 1.4 | 1.5 ± 1.4 | 1.5 ± 1.4 |
| cellulose ether | | 7.0 ± 6.5 | | | 7.0 ± 6.5 | 7.0 ± 6.5 | | 7.0 ± 6.5 |
| cross-linked polymer | | | 7.0 ± 6.5 | | 7.0 ± 6.5 | | 7.0 ± 6.5 | 7.0 ± 6.5 |
| binder | | | | 7.0 ± 6.5 | | 7.0 ± 6.5 | 7.0 ± 6.5 | 7.0 ± 6.5 |
| Ingredient [wt.-%] | A⁹ | A¹⁰ | A¹¹ | A¹² | A¹³ | A¹⁴ | A¹⁵ | A¹⁶ |
| pseudoephedrine or a salt | 30 ± 25 | 30 ± 25 | 30 ± 25 | 30 ± 25 | 30 ± 25 | 30 ± 25 | 30 ± 25 | 30 ± 25 |
| thereof | | | | | | | | |
| polyethylene oxide | 45 ± 25 | 45 ± 25 | 45 ± 25 | 45 ± 25 | 45 ± 25 | 45 ± 25 | 45 ± 25 | 45 ± 25 |
| polyethylene glycol | 10 ± 9.0 | 10 ± 9.0 | 10 ± 9.0 | 10 ± 9.0 | 10 ± 9.0 | 10 ± 9.0 | 10 ± 9.0 | 10 ± 9.0 |
| α-tocopherol | 1.5 ± 1.4 | 1.5 ± 1.4 | 1.5 ± 1.4 | 1.5 ± 1.4 | 1.5 ± 1.4 | 1.5 ± 1.4 | 1.5 ± 1.4 | 1.5 ± 1.4 |
| HPMC | | 7.0 ± 6.5 | | | 7.0 ± 6.5 | 7.0 ± 6.5 | | 7.0 ± 6.5 |
| croscarmellose sodium | | | 7.0 ± 6.5 | | 7.0 ± 6.5 | | 7.0 ± 6.5 | 7.0 ± 6.5 |
| polyvinylpyrrolidone | | | | 7.0 ± 6.5 | | 7.0 ± 6.5 | 7.0 ± 6.5 | 7.0 ± 6.5 |
| Ingredient [wt.-%] | A¹⁷ | A¹⁸ | A¹⁹ | A²⁰ | A²¹ | A²² | A²³ | A²⁴ |
| ephedrine component | 30 ± 17 | 30 ± 17 | 30 ± 17 | 30 ± 17 | 30 ± 17 | 30 ± 17 | 30 ± 17 | 30 ± 17 |
| polyalkylene oxide | 45 ± 18 | 45 ± 18 | 45 ± 18 | 45 ± 18 | 45 ± 18 | 45 ± 18 | 45 ± 18 | 45 ± 18 |
| plasticizer | 10 ± 6.0 | 10 ± 6.0 | 10 ± 6.0 | 10 ± 6.0 | 10 ± 6.0 | 10 ± 6.0 | 10 ± 6.0 | 10 ± 6.0 |
| antioxidant | 1.5 ± 1.0 | 1.5 ± 1.0 | 1.5 ± 1.0 | 1.5 ± 1.0 | 1.5 ± 1.0 | 1.5 ± 1.0 | 1.5 ± 1.0 | 1.5 ± 1.0 |
| cellulose ether | | 7.0 ± 4.0 | | | 7.0 ± 4.0 | 7.0 ± 4.0 | | 7.0 ± 4.0 |
| cross-linked polymer | | | 7.0 ± 4.0 | | 7.0 ± 4.0 | | 7.0 ± 4.0 | 7.0 ± 4.0 |
| binder | | | | 7.0 ± 4.0 | | 7.0 ± 4.0 | 7.0 ± 4.0 | 7.0 ± 4.0 |
| Ingredient [wt.-%] | A²⁵ | A²⁶ | A²⁷ | A²⁸ | A²⁹ | A³⁰ | A³¹ | A³² |
| pseudoephedrine or a salt | 30 ± 17 | 30 ± 17 | 30 ± 17 | 30 ± 17 | 30 ± 17 | 30 ± 17 | 30 ± 17 | 30 ± 17 |
| thereof | | | | | | | | |
| polyethylene oxide | 45 ± 18 | 45 ± 18 | 45 ± 18 | 45 ± 18 | 45 ± 18 | 45 ± 18 | 45 ± 18 | 45 ± 18 |
| polyethylene glycol | 10 ± 6.0 | 10 ± 6.0 | 10 ± 6.0 | 10 ± 6.0 | 10 ± 6.0 | 10 ± 6.0 | 10 ± 6.0 | 10 ± 6.0 |
| α-tocopherol | 1.5 ± 1.0 | 1.5 ± 1.0 | 1.5 ± 1.0 | 1.5 ± 1.0 | 1.5 ± 1.0 | 1.5 ± 1.0 | 1.5 ± 1.0 | 1.5 ± 1.0 |
| HPMC | | 7.0 ± 4.0 | | | 7.0 ± 4.0 | 7.0 ± 4.0 | | 7.0 ± 4.0 |
| croscarmellose sodium | | | 7.0 ± 4.0 | | 7.0 ± 4.0 | | 7.0 ± 4.0 | 7.0 ± 4.0 |
| polyvinylpyrrolidone | | | | 7.0 ± 4.0 | | 7.0 ± 4.0 | 7.0 ± 4.0 | 7.0 ± 4.0 |
| Ingredient [wt.-%] | A³³ | A³⁴ | A³⁵ | A³⁶ | A³⁷ | A³⁸ | A³⁹ | A⁴⁰ |
| ephedrine component | 30 ± 9 | 30 ± 9 | 30 ± 9 | 30 ± 9 | 30 ± 9 | 30 ± 9 | 30 ± 9 | 30 ± 9 |
| polyalkylene oxide | 45 ± 10 | 45 ± 10 | 45 ± 10 | 45 ± 10 | 45 ± 10 | 45 ± 10 | 45 ± 10 | 45 ± 10 |
| plasticizer | 10 ± 3.0 | 10 ± 3.0 | 10 ± 3.0 | 10 ± 3.0 | 10 ± 3.0 | 10 ± 3.0 | 10 ± 3.0 | 10 ± 3.0 |
| antioxidant | 1.5 ± 0.5 | 1.5 ± 0.5 | 1.5 ± 0.5 | 1.5 ± 0.5 | 1.5 ± 0.5 | 1.5 ± 0.5 | 1.5 ± 0.5 | 1.5 ± 0.5 |
| cellulose ether | | 7.0 ± 2.0 | | | 7.0 ± 2.0 | 7.0 ± 2.0 | | 7.0 ± 2.0 |
| cross-linked polymer | | | 7.0 ± 2.0 | | 7.0 ± 2.0 | | 7.0 ± 2.0 | 7.0 ± 2.0 |
| binder | | | | 7.0 ± 2.0 | | 7.0 ± 2.0 | 7.0 ± 2.0 | 7.0 ± 2.0 |
| Ingredient [wt.-%] | A⁴¹ | A⁴² | A⁴³ | A⁴⁴ | A⁴⁵ | A⁴⁶ | A⁴⁷ | A⁴⁸ |
| pseudoephedrine or a salt | 30 ± 9 | 30 ± 9 | 30 ± 9 | 30 ± 9 | 30 ± 9 | 30 ± 9 | 30 ± 9 | 30 ± 9 |
| thereof | | | | | | | | |
| polyethylene oxide | 45 ± 10 | 45 ± 10 | 45 ± 10 | 45 ± 10 | 45 ± 10 | 45 ± 10 | 45 ± 10 | 45 ± 10 |
| polyethylene glycol | 10 ± 3.0 | 10 ± 3.0 | 10 ± 3.0 | 10 ± 3.0 | 10 ± 3.0 | 10 ± 3.0 | 10 ± 3.0 | 10 ± 3.0 |
| α-tocopherol | 1.5 ± 0.5 | 1.5 ± 0.5 | 1.5 ± 0.5 | 1.5 ± 0.5 | 1.5 ± 0.5 | 1.5 ± 0.5 | 1.5 ± 0.5 | 1.5 ± 0.5 |
| HPMC | | 7.0 ± 2.0 | | | 7.0 ± 2.0 | 7.0 ± 2.0 | | 7.0 ± 2.0 |
| croscarmellose sodium | | | 7.0 ± 2.0 | | 7.0 ± 2.0 | | 7.0 ± 2.0 | 7.0 ± 2.0 |
| polyvinylpyrrolidone | | | | 7.0 ± 2.0 | | 7.0 ± 2.0 | 7.0 ± 2.0 | 7.0 ± 2.0 |

In the above tables, all percentages are weight percent relative to the total weight of the pharmaceutical dosage form.

Preferably, the pharmaceutical dosage form according to the invention is prepared by hot-melt extrusion.

Preferably, the pharmaceutical dosage form according to the invention is prepared by thermoforming, although also other methods of thermoforming may be used in order to manufacture the pharmaceutical dosage form according to the invention such as press-molding at elevated temperature or heating of tablets that were manufactured by conventional compression in a first step and then heated above the softening temperature of the polymer in the tablet in a second step to form hard tablets. In this regards, thermoforming means the forming or molding of a mass after the application of heat. In a preferred embodiment, the pharmaceutical dosage form is thermoformed by hot-melt extrusion.

In a preferred embodiment, the mixture of ingredients is heated and subsequently compressed under conditions (time, temperature and pressure) sufficient in order to achieve the desired mechanical properties, e.g. in terms of breaking strength and the like. This technique may be achieved e.g. by means of a tabletting tool which is either heated and/or which is filled with the heated mixture that is subsequently compressed without further supply of heat or with simultaneous additional supply of heat.

In another preferred embodiment, the mixture of ingredients is heated and simultaneously compressed under conditions (time, temperature and pressure) sufficient in order to achieve the desired mechanical properties, e.g. in terms of breaking strength and the like. This technique may be achieved e.g. by means of an extruder with one or more heating zones, wherein the mixture is heated and simultaneously subjected to extrusion forces finally resulting in a compression of the heated mixture.

In still another embodiment, the mixture of ingredients is compressed under ambient conditions at sufficient pressure and subsequently heated (cured) under conditions (time, temperature) sufficient in order to achieve the desired mechanical properties, e.g. in terms of breaking strength and the like. This technique may be achieved e.g. by means of a curing oven in which the compressed articles are cured for a sufficient time at a sufficient temperature, preferably without exerting any further pressure. Such process is further described e.g. in US 2009/0081290.

A particularly preferred process for the manufacture of the particles according to the invention involves hot-melt extrusion. In this process, the particles according to the invention are produced by thermoforming with the assistance of an extruder, preferably without there being any observable consequent discoloration of the extrudate.

In a preferred embodiment, the pharmaceutical dosage form is prepared by hot melt-extrusion, preferably by means of a twin-screw-extruder. Melt extrusion preferably provides a melt-extruded strand that is preferably cut into monoliths, which are then compressed and formed into tablets. In this regard, the term “tablets” is preferably not to be understood as pharmaceutical dosage forms being made by compression of powder or granules (compressi) but rather, as shaped extrudates. Preferably, compression is achieved by means of a die and a punch, preferably from a monolithic mass obtained by melt extrusion. If obtained via melt extrusion, the compressing step is preferably carried out with a monolithic mass exhibiting ambient temperature, that is, a temperature in the range from 20 to 25° C. The strands obtained by way of extrusion can either be subjected to the compression step as such or can be cut prior to the compression step. This cutting can be performed by usual techniques, for example using rotating knives or compressed air. Alternatively, the shaping can take place as described in EP-A 240 906 by the extrudate being passed between two counter-rotating calender rolls and being shaped directly to tablets. It is of course also possible to subject the extruded strands to the compression step or to the cutting step when still warm, that is more or less immediately after the extrusion step. The extrusion is preferably carried out by means of a twin-screw extruder.

The pharmaceutical dosage form according to the invention may be produced by different processes, the particularly preferred of which are explained in greater detail below. Several suitable processes have already been described in the prior art. In this regard it can be referred to, e.g., WO 2005/016313, WO 2005/016314, WO 2005/063214, WO 2005/102286, WO 2006/002883, WO 2006/002884, WO 2006/002886, WO 2006/082097, and WO 2006/082099.

The present invention also relates to pharmaceutical dosage forms that are obtainable by any of the processes described here below.

In general, the process for the production of the pharmaceutical dosage form according to the invention preferably comprises the following steps:

mixing all ingredients;
optionally pre-forming the mixture obtained from step (a), preferably by applying heat and/or force to the mixture obtained from step (a), the quantity of heat supplied preferably not being sufficient to heat the polyalkylene oxide up to its softening point;
hardening the mixture by applying heat and force, and after the process decreasing heat and force, it being possible to supply the heat during and/or before the application of force and the quantity of heat supplied being sufficient to heat the polyalkylene oxide at least up to its softening point;
optionally singulating the hardened mixture;
optionally shaping the pharmaceutical dosage form; and
optionally providing a film coating.

Heat may be supplied directly, e.g. by contact or by means of hot gas such as hot air, or with the assistance of ultrasound; or is indirectly supplied by friction and/or shear. Force may be applied and/or the pharmaceutical dosage form may be shaped for example by direct tabletting or with the assistance of a suitable extruder, particularly by means of a screw extruder equipped with two screws (twin-screw-extruder) or by means of a planetary gear extruder.

The final shape of the pharmaceutical dosage form may either be provided during the hardening of the mixture by applying heat and force (step (c)) or in a subsequent step (step (e)). In both cases, the mixture of all components is preferably in the plastified state, i.e. preferably, shaping is performed at a temperature at least above the softening point of the polyalkylene oxide. However, extrusion at lower temperatures, e.g. ambient temperature, is also possible and may be preferred.

Shaping can be performed, e.g., by means of a tabletting press comprising die and punches of appropriate shape.

A particularly preferred process for the manufacture of the pharmaceutical dosage form of the invention involves hot-melt extrusion. In this process, the pharmaceutical dosage form according to the invention is produced by thermoforming with the assistance of an extruder, preferably without there being any observable consequent discoloration of the extrudate.

This process is characterized in that

a) all components are mixed,
b) the resultant mixture is heated in the extruder at least up to the softening point of the polyalkylene oxide and extruded through the outlet orifice of the extruder by application of force,
c) the still plastic extrudate is singulated and formed into the pharmaceutical dosage form or
d) the cooled and optionally reheated singulated extrudate is formed into the pharmaceutical dosage form.

Mixing of the components according to process step a) may also proceed in the extruder.

The components may also be mixed in a mixer known to the person skilled in the art. The mixer may, for example, be a roll mixer, shaking mixer, shear mixer or compulsory mixer.

The, preferably molten, mixture which has been heated in the extruder at least up to the softening point of polyalkylene oxide is extruded from the extruder through a die with at least one bore.

The process according to the invention requires the use of suitable extruders, preferably screw extruders. Screw extruders which are equipped with two screws (twin-screw-extruders) are particularly preferred.

The extrusion is preferably performed so that the expansion of the strand due to extrusion is not more than 30%, i.e. that when using a die with a bore having a diameter of e.g. 6 mm, the extruded strand should have a diameter of not more than 8 mm. More preferably, the expansion of the strand is not more than 25%, still more preferably not more than 20%, most preferably not more than 15% and in particular not more than 10%.

Preferably, extrusion is performed in the absence of water, i.e., no water is added. However, traces of water (e.g., caused by atmospheric humidity) may be present.

The extruder preferably comprises at least two temperature zones, with heating of the mixture at least up to the softening point of the polyalkylene oxide preceding in the first zone, which is downstream from a feed zone and optionally mixing zone. The throughput of the mixture is preferably from 1.0 kg to 15 kg/hour. In a preferred embodiment, the throughput is from 1 to 3.5 kg/hour. In another preferred embodiment, the throughput is from 4 to 15 kg/hour.

In a preferred embodiment, the die head pressure is within the range of from 25 to 100 bar. The die head pressure can be adjusted inter alia by die geometry, temperature profile and extrusion speed.

The die geometry or the geometry of the bores is freely selectable. The die or the bores may accordingly exhibit a round, oblong or oval cross-section, wherein the round cross-section preferably has a diameter of 0.1 mm to 15 mm and the oblong cross-section preferably has a maximum lengthwise extension of 21 mm and a crosswise extension of 10 mm. Preferably, the die or the bores have a round cross-section. The casing of the extruder used according to the invention may be heated or cooled. The corresponding temperature control, i.e. heating or cooling, is so arranged that the mixture to be extruded exhibits at least an average temperature (product temperature) corresponding to the softening temperature of the polyalkylene oxide and does not rise above a temperature at which the ephedrine component to be processed may be damaged. Preferably, the temperature of the mixture to be extruded is adjusted to below 180° C., preferably below 150° C., but at least to the softening temperature of polyalkylene oxide. Typical extrusion temperatures are 120° C. and 130° C.

In a preferred embodiment, the extruder torque is within the range of from 30 to 95%. Extruder torque can be adjusted inter alia by die geometry, temperature profile and extrusion speed.

After extrusion of the molten mixture and optional cooling of the extruded strand or extruded strands, the extrudates are preferably singulated. This singulation may preferably be performed by cutting up the extrudates by means of revolving or rotating knives, water jet cutters, wires, blades or with the assistance of laser cutters.

Preferably, intermediate or final storage of the optionally singulated extrudate or the final shape of the pharmaceutical dosage form according to the invention is performed under oxygen-free atmosphere which may be achieved, e.g., by means of oxygen-scavengers.

The singulated extrudate may be press-formed into tablets in order to impart the final shape to the pharmaceutical dosage form.

The application of force in the extruder onto the at least plasticized mixture is adjusted by controlling the rotational speed of the conveying device in the extruder and the geometry thereof and by dimensioning the outlet orifice in such a manner that the pressure necessary for extruding the plasticized mixture is built up in the extruder, preferably immediately prior to extrusion. The extrusion parameters which, for each particular composition, are necessary to give rise to a pharmaceutical dosage form with desired mechanical properties, may be established by simple preliminary testing.

For example but not limiting, extrusion may be performed by means of a twin-screw-extruder type ZSE 18 or ZSE27 (Leistritz, Nurnberg, Germany), screw diameters of 18 or 27 mm. Screws having eccentric ends may be used. A heatable die with a round bore having a diameter of 7, 8, or 9 mm may be used. The extrusion parameters may be adjusted e.g. to the following values: rotational speed of the screws: 120 Upm; delivery rate 2 kg/h for a ZSE 18 or 8 kg/h for a ZSE27; product temperature: in front of die 125° C. and behind die 135° C.; and jacket temperature: 110° C.

Preferably, extrusion is performed by means of twin-screw-extruders or planetary-gear-extruders, twin-screw extruders (co-rotating or contra-rotating) being particularly preferred.

The process for the preparation of the pharmaceutical dosage form according to the invention is preferably performed continuously. Preferably, the process involves the extrusion of a homogeneous mixture of all components. It is particularly advantageous if the thus obtained intermediate, e.g. the strand obtained by extrusion, exhibits uniform properties. Particularly desirable are uniform density, uniform distribution of the active ingredient, uniform mechanical properties, uniform porosity, uniform appearance of the surface, etc. Only under these circumstances the uniformity of the pharmacological properties, such as the stability of the release profile, may be ensured and the amount of rejects can be kept low.

Another aspect of the invention relates to a pharmaceutical dosage form according to the invention for use in the treatment of a disease, disorder or condition preferably selected from the group consisting of tissue hyperemia, edema, and nasal congestion.

A further aspect of the invention relates to the use of an ephedrine component for the manufacture of a pharmaceutical dosage form according to the invention for the treatment of a disease, disorder or condition preferably selected from the group consisting of tissue hyperemia, edema, and nasal congestion.

A still further aspect of the invention relates to a method for the treatment of a disease, disorder or condition preferably selected from the group consisting of tissue hyperemia, edema, and nasal congestion, comprising the administration of the pharmaceutical dosage form according to the invention to a subject in need thereof.

Preferably, the pharmaceutical dosage form for use according to the invention is administered orally. Preferably, the subject is a human.

Further, the invention relates to a method for the prophylaxis and/or the treatment of a disease, disorder or condition preferably selected from the group consisting of tissue hyperemia, edema, and nasal congestion, the method comprising the provision or administration of the pharmaceutical dosage form according to the invention, thereby preventing chemical conversion of the ephedrine component to methamphetamine, particularly involving comminution of the pharmaceutical dosage form by mechanical action and/or solvent extraction. Preferably, the mechanical action is selected from the group consisting of grinding in a mortar, pounding, and using apparatuses for pulverizing conventional pharmaceutical dosage forms.

Another aspect of the invention relates to the use of a pharmaceutical dosage form according to the invention s described above for impeding or preventing the chemical conversion of the ephedrine component to methamphetamine or a physiologically acceptable salt thereof.

The following examples further illustrate the invention but are not to be construed as limiting its scope:

### EXAMPLE 1—PREPARATION OF CUT RODS

### a) Inventive Formulations

Cut rods having the following composition were manufactured by hot-melt extrusion:

| Ingredient [wt.-%] | A | B | C | D | E | F |
|---|---|---|---|---|---|---|
| Pseudoephedrine HCl | 30.00 | 30.00 | 30.00 | 30.00 | 30.00 | 30.00 |
| Polyethylene oxide Polyox | 50.72 | 45.07 | 44.03 | 38.30 | 44.03 | 44.03 |
| WSR 303 NF | | | | | | |
| Polyethylene glycol | 12.08 | 10.73 | 10.48 | 9.14 | 10.48 | 10.48 |
| Polyglycol 6000 PF | | | | | | |
| α-Tocopherol | 0.20 | 0.20 | 1.49 | 1.49 | 1.49 | 1.49 |
| HPMC K100 M Premium | 7.00 | 7.00 | 7.00 | 7.00 | 7.00 | — |
| Croscarmellose SD 711 | — | — | — | 7.00 | 7.00 | 7.00 |
| Polyvinylpyrrolidone PVP | — | 7.00 | 7.00 | 7.00 | — | 7.00 |
| K30 | | | | | | |

Each cut rod had a total weight of 400 mg.

Pseudoephedrine hydrochloride, polyethylene oxide, polyethylene glycol, α-tocopherol, and all other ingredients were weighted and sieved. The powder was mixed and dosed gravimetrically to an extruder. Hot-melt extrusion was performed by means of a twin screw extruder of type ZSE 18 (Leistritz, Nurnberg, Germany) that was equipped with a heatable round die having a diameter of 5 mm.

The hot extrudate was cooled on a conveyor belt and the cooled extrusion strand was comminuted to cut rods.

### b) Comparative Formulations

The inventive Formulations A to F were compared with three commercial preparations of pseudoephedrine that are said to provide tamper resistance, namely Sudafed® (Comparator A), Nexafed® (Comparator B), and Zephrex-D® Softgels (Comparator C).

### EXAMPLE 2—TAMPER RESISTANCE WITH RESPECT TO MECHANICAL STRENGTH OF CUT RODS

The mechanical properties of the cut rods obtained in Example 1 were tested with various means that are conventionally used for disruption including hammer, coffee mill, spoons, knife, mortar, PedEgg® (calosity plane, corn parer), pliers, and the like.

PedEgg® provided the most efficient means of disrupting the cut rods. Nonetheless, sieve analysis revealed that at least 95 wt.-% of the resultant fragments were not smaller than 0.21 mm, and 45 to 88 wt.-% of the resultant fragments were larger than 1 mm.

Thus, all cut rods had an increased breaking strength.

### EXAMPLE 3—BIOAVAILABILITY AND DISSOLUTION IN AQUEOUS MEDIA

All cut rods according to inventive Formulations A to F provided prolonged release of pseudoephedrine under in vitro conditions.

In water and also in aqueous ethanol, the disrupted cut rods provided a similar dissolution like Comparators A and B.

From the results in water it can be concluded that the inventive cut rods according to Formulations A to F should have a similar or the same bioavailability as Comparators A and B.

### EXAMPLE 4—TAMPER RESISTANCE WITH RESPECT TO CHEMICAL CONVERTIBILITY INTO METHAMPHETAMINE

The tamper resistance of the cut rods obtained in Example 1 was tested with respect to chemical conversion of pseudoephedrine to methamphetamine. Both approaches were investigated, a) chemical conversion according to the one pot procedure, as well as b) chemical conversion after extraction with organic solvents.

### a) One Pot Conversion (Shake and Bake One Pot Procedure)

It was investigated whether methamphetamine could be synthesized from pseudoephedrine by direct one-pot conversion (reduction with lithium). For this purpose, the cut rods of Formulations A and F were mechanically disrupted by means of a PedEgg®, whereas the cut rods of Formulations B, C, D and E were mechanically disrupted by means of a commercial coffee grinder.

The softgels of Comparator C were ground in a coffee grinder. The particle size of the softgels was reduced to a large degree using a coffee grinder for about 30 seconds. Grinding produced a thick, waxy substance that had to be scraped from the sides of the coffee grinder. Portions of unground softgels remained, but overall the ground material was useful for the purposes of conducting the one-pot experiments.

In order to simulate the shake and bake one pot procedure, the thus obtained material was mixed with an ether/hexane mixture in a plastic pressure pop bottle. Ammonium nitrate, lithium, sodium hydroxide and a small volume of water were added and the bottle was closed (see also R. Turkington, Chemicals Used For Illegal Purposes, A Guide for First Responders to Identify Explosives, Recreational Drugs, and Poisons, John Wiley & Sons 2010, page 247). The container was shaken and periodically, the developed pressure was released and further sodium hydroxide was added. The tests were performed at NMS labs (US) according to an established standard procedure.

The course of the reaction was monitored by LC-MS. Aliquoted samples were taken at various time points and diluted for analysis.

The results are shown in the table here below:

| | | Methamphetamine | Pseudoephedrine |
|---|---|---|---|
| Formulation | after | Produced (%) | Recovered (wt.-%) |
| A | 30 min | 0.4 | 12.4 |
| | 60 min | 0.4 | 15.9 |
| | 90 min | 0.5 | 20.8 |
| B | 30 min | 0.3 | 12.4 |
| | 60 min | 0.0 | 11.8 |
| | 90 min | 1.0 | 25.8 |
| C | 30 min | 0.0 | 9.1 |
| | 60 min | 0.3 | 9.4 |
| | 90 min | 0.3 | 13.4 |
| D | 30 min | 0.3 | 9.9 |
| | 60 min | 0.3 | 14.2 |
| | 90 min | 0.8 | 21.8 |
| E | 30 min | 0.3 | 11.3 |
| | 60 min | 0.4 | 12.2 |
| | 90 min | 0.4 | 21.2 |
| F | 30 min | 0.0 | 7.5 |
| | 60 min | 0.0 | 13.3 |
| | 90 min | 0.3 | 14.2 |
| Comparator A | 60 min | 43.4 | 38.2 |
| (Sudafed ®) | 120 min | 58.8 | 1.3 |
| | 180 min | 63.8 | 0.1 |
| Comparator B | 60 min | 8.4 | 55.2 |
| (Nexafed ®) | 120 min | 58.3 | 15.2 |
| | 180 min | 61.9 | 0.8 |
| Comparator C | 60 min | 36.3 | 23.0 |
| (Zephrex-D ®) | 120 min | 42.4 | 3.7 |
| | 180 min | 48.5 | 4.2 |

Reactions for inventive formulations A-F were relatively “violent”; on multiple occasions, flare ups were observed and there was significant pressure buildup and smoking as the reaction proceeded. As a result, for the sake of safety, these reactions were carried out to only 1.5 hours. The Comparator reactions were milder reactions and were carried out to 3 hours.

Additionally, the cut rod material that settled at the bottom of the reaction vessel seemed to capture the lithium, creating an aggregate when the second strip was added and the reaction proceeded for a while. The material expanded when pressure was released and went back into solution when the container was re-pressurized.

As demonstrated by the above comparative data, compared to comparators A, B and C the intact cut rods according to the invention provide a substantially improved resistance against conversion of pseudoephedrine into methamphetamine by direct one-pot conversion. Comparing the inventive formulations A-F with comparators A, B and C, essentially no methamphetamine was produced using the inventive formulations; instead, pseudoephedrine was released over time unlike the decrease in pseudoephedrine concentration over time that was observed in the analyses of comparators A, B and C.

### b) Chemical Conversion of Extracted Material

It was also investigated whether methamphetamine could be synthesized from pseudoephedrine after extraction from mechanically disrupted material (PedEgg®) with various organic solvents.

For that purpose, the extractability of pseudoephedrine was tested in various solvents including diethyl ether:water, ethyl acetate:water and methylene chloride:water at various relative ratios and pH ranges. In most of the diethylether extractions, viscous material blocked the opening of the separatory funnel, making it difficult to drain the aqueous layer. Overall, the extraction experiments revealed that pseudoephedrine extracted more efficiently into methylene chloride and in few cases into ethyl acetate.

Inventive formulations C, D and E as well comparators A and B were grounded and extracted with the appropriate solvent. The extracts were dried and it was tested whether methamphetamine could be synthesized from the extracted pseudoephedrine by reduction with lithium in analogy to the above shake and bake one pot procedure.

The results are shown in the table here below:

| | | Methamphetamine | Pseudoephedrine |
|---|---|---|---|
| Formulation | Solvent | Produced (%) | Recovered (wt.-%) |
| D | diethyl ether | 0.0 | 0.5 |
| D | methylene | 1.1 | 22.8 |
| | chloride | | |
| C | methylene | 7.1 | 41.7 |
| | chloride | | |
| E | methylene | 0.6 | 36.0 |
| | chloride | | |
| E | ethyl acetate | 2.7 | 5.9 |
| Comparator A | methylene | 54.0 | 58.7 |
| | chloride | | |
| Comparator B | methylene | 10.8 | 5.9 |
| | chloride | | |

Comparable to the one pot conversion experiments, the inventive formulations C, D and E gave in all cases less methamphetamine than comparators A and B.

1. A pharmaceutical dosage form having a breaking strength of at least 300 N and comprising an ephedrine component selected from the group consisting of ephedrine, pseudoephedrine and the physiologically acceptable salts thereof, wherein the weight content of the ephedrine component is within the range of from 0.1 to 60 wt.-%, relative to the total weight of the pharmaceutical dosage form.

2. The pharmaceutical dosage form according to claim 1, wherein the ephedrine component comprises pseudoephedrine hydrochloride or pseudoephedrine sulfate.

3. The pharmaceutical dosage form according to claim 1, wherein the weight content of the ephedrine component is within the range of from 10 to 50 wt.-%, relative to the total weight of the pharmaceutical dosage form.

4. (canceled)

5. The pharmaceutical dosage form according to claim 1, which comprises a polyalkylene oxide.

6. (canceled)

7. The pharmaceutical dosage form according to claim 5, wherein the polyalkylene oxide has a weight average molecular weight of at least 200,000 g/mol.

8. (canceled)

9. (canceled)

10. (canceled)

11. (canceled)

12. (canceled)

13. (canceled)

14. (canceled)

15. (canceled)

16. The pharmaceutical dosage form according to claim 1, which comprises an antioxidant.

17. The pharmaceutical dosage form according to claim 16, wherein the antioxidant is selected from the group consisting of ascorbic acid, salts of ascorbic acid, butylhydroxyanisole, butylhydroxytoluene, monothioglycerol, phosphorous acid, α-tocopherol, α-tocopheryl acetate, coniferyl benzoate, nordihydroguajaretic acid, gallus acid esters, and sodium bisulfate.

18. The pharmaceutical dosage form according to claim 17, wherein the antioxidant is α-tocopherol.

19. (canceled)

20. (canceled)

21. (canceled)

22. The pharmaceutical dosage form according to claim 1, which comprises a cellulose ether.

23. The pharmaceutical dosage form according to claim 22, wherein the cellulose ether is selected from the group consisting of methyl cellulose, ethyl cellulose, propyl cellulose, hydroxyethyl cellulose, hydroxypropyl cellulose, hydroxypropylmethyl cellulose, carboxymethyl cellulose, salts of carboxymethyl cellulose, and mixtures of any of the foregoing.

24. The pharmaceutical dosage form according to claim 23, wherein the cellulose ether is hydroxypropylmethyl cellulose.

25. (canceled)

26. (canceled)

27. (canceled)

28. (canceled)

29. (canceled)

30. (canceled)

31. The pharmaceutical dosage form according to claim 1, which comprises a binder.

32. The pharmaceutical dosage form according to claim 31, wherein the binder is selected from the group consisting of disaccharides, starch, modified starch, sugar alcohols, polyvinylpyrrolidone, and mixtures of any of the foregoing.

33. (canceled)

34. (canceled)

35. (canceled)

36. (canceled)

37. (canceled)

38. (canceled)

39. (canceled)

40. The pharmaceutical dosage form according to claim 1, which comprises a cross-linked polymer.

41. The pharmaceutical dosage form according to claim 40, wherein the cross-linked polymer is selected from the group consisting of croscarmellose, salts or croscarmellose, crospovidone, and mixtures of any of the foregoing.

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. The pharmaceutical dosage form according to claim 1, which provides resistance against extracting the ephedrine component from the pharmaceutical dosage form by means of aqueous or organic solvents.

56. The pharmaceutical dosage form according to claim 1, wherein
the weight content of the ephedrine component is within the range of from 10 to 50 wt.-%, relative to the total weight of the pharmaceutical dosage form; and
the pharmaceutical dosage form comprises a polyalkylene oxide, wherein
the weight content of the polyalkylene oxide is within the range of from 25 to 65 wt.-%, relative to the total weight of the pharmaceutical dosage form; and/or
the relative weight ratio of the polyalkylene oxide to the ephedrine component is within the range of from 3:1 to 1:2; and/or
the polyalkylene oxide has a weight average molecular weight of at least 500,000 g/mol; and
the pharmaceutical dosage form comprises an antioxidant, wherein
the weight content of the antioxidant is at least 0.5 wt.-%, relative to the total weight of the pharmaceutical dosage form; and/or
the relative weight ratio of the ephedrine component to the antioxidant is within the range of from 5:1 to 35:1; and
the pharmaceutical dosage form comprises a binder, wherein
the weight content of the binder is within the range of from 0.5 to 20 wt.-%, relative to the total weight of the pharmaceutical dosage form; and/or
the relative weight ratio of the ephedrine component to the binder is within the range of from 3:1 to 5.5:1.

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. A method for treating a disease, disorder or condition selected from the group consisting of tissue hyperemia, edema, and nasal congestion, said method comprising administering to a patient in need of such treating the pharmaceutical dosage form according to claim 1.

72. The method according to claim 71, wherein the pharmaceutical dosage form is administered orally.

73. The method according to claim 71, wherein the pharmaceutical dosage form is administered once daily, twice daily or thrice daily.
